(19) United States
(12) Patent Application Publication
(10) Pub. No.: US 2011/0240504 A1
(43) Pub. Date: Oct. 6, 2011

(54) METHOD AND APPARATUS FOR PACKAGING AND DISPENSING PHARMACEUTICALS

(75) Inventors: Arturo Tramonte, Brampton (CA); Maria L. Tagliabracci, Aurora (CA); Ryan W. Unwin, Markham (CA)

(73) Assignee: APOTEX TECHNOLOGIES INC., Toronto, ON (CA)

(21) Appl. No.: 13/132,859

(22) PCT Filed: Dec. 3, 2009
(86) PCT No.: PCT/CA2009/001761

§ 371 Date: Jun. 3, 2011

(30) Foreign Application Priority Data
Dec. 3, 2008 (CA) 2653011

(51) Int. Cl. B65D 73/00; A61K 31/40; A61K 31/522; B65B 5/10

(52) U.S. Cl. 206/489; 514/423; 514/263.38; 53/475

(57) ABSTRACT

A method of packaging one or more pharmaceuticals comprising separating a plurality of unit doses into a plurality of unit dose compartments, wherein each unit dose compartment of the plurality of unit dose compartments holds only one unit dose of the plurality of unit doses, each unit dose having the same strength, wherein the plurality of unit doses is precisely a quantity equal to a most commonly prescribed number of unit doses in individual prescriptions for the unit dose, or a multiple thereof is provided. Also provided are kits prepared by the method. Methods for designing such kits and packaging are also provided.

### TECHNICAL FIELD

This invention relates to the dispensing and packaging of pharmaceuticals and more particularly to dispensing and packaging of pharmaceuticals by a manufacturer for use by a pharmacist.

### BACKGROUND

Unit doses of pharmaceuticals are sold in packages that include 10's or 100's of tablets and such packages are used by pharmacists to dispense the prescribed number of tablets to a patient. For example, Atorvastatin is sold in unit doses of 10 mg, 20 mg, 40 mg and 80 mg, each of which are sold in 90 pill bottles. Another, example is Valacyclovir, which is sold in unit doses of 500 mg and 1000 mg. 500 mg valacyclovir is sold in 42-pill bottles and 100-pill bottles, while 1000 mg valacyclovir is sold in 21-pill blister packs.

Pharmacists count and separate the appropriate number of pills at the appropriate strength depending on the prescription written by a physician for a particular patient and then provide the counted and separated pills to the patient in a separate package, typically a plastic bottle affixed with a specific patient label.

Despite the advantages of bottle dispensation, there are also problems associated with bottle dispensation. Pills dispensed (by a pharmacist or a customer thereof) from a bottle can result in “runaway” pills. This is often termed spillage. Furthermore, the pills in a bottle often break or otherwise perish faster than other forms of packaging, such as blister packaging. When a retail pharmacist returns open but unused pills to a pharmaceutical company, the company cannot re-distribute those pills and the pills are typically thrown out. Nevertheless, the company may still issue a return credit to the pharmacist.

Furthermore, bottles are often difficult to transport and store. The typical shapes of bottles results in poor stacking/packing characteristics. For example, they often require more shelf space as round bottles often result in wasted spaces appearing between touching bottles having curved sides. Round bottles are also subject to rolling and are often more inconvenient to handle than other forms of packaging. For example, blister packs may lay flat, may be stacked, and moreover, the patient may obtain the original packaging from the pharmaceutical company.

Many drugs are subject to ambient conditions and thus become unstable during long shelf life. To improve shelf life a drug may be packaged in blister packs to protect the drug. In addition, naked unit doses of pills in a bottle are at risk for contamination once opened at the retail level and such a risk is increased each time the bottle is opened at the retail level.

Unit doses in blister packaging are difficult to separate from one another and often require the use of a cutting tool, particularly if the blister cavities are not separated by perforations. This is particularly problematic for a weakened, infirm, or senior patient where the patient's strength or dexterity is compromised. This makes dispensing of the correct number of unit doses more problematic as well as the possible loss of pills due to ripping of the lidding foil when tearing the blister sheet. Furthermore, breaking blister sheets often results in partial blister sheets with unusual numbers of unit doses remaining that may be problematic for a pharmacist to make use of.

### SUMMARY

This invention is based, in part, on prescription practices, dispensing practices and an elucidation of the understanding of an intersection between these two areas of practice. In particular, the present invention provides methods and kits for dispensing one or more particular pharmaceuticals such that the benefits of one method of packaging may be provided in combination with the benefits of another method of packaging at the same time as eliminating many of the drawbacks associated with handling a naked unit dose. For example, the benefits provided by blister packaging in terms of dose integrity and preservation may be combined with the benefits of separating unit doses from a bottle containing multiple unit doses in terms of ease of providing the appropriate number of unit doses. The present invention may reduce the need to cut blister packaging or reduce the loss, breakage or contamination of unit doses associated with handling naked unit doses or tearing blister packaging.

There are many reasons that a physician does not want to write a prescription for too many doses of a chronically prescribed medication for an individual patient. Some of these reasons include monitoring patient compliance, reducing grey and black market pharmaceutical trade, shelf-life of the pharmaceutical as well as many others. Consequently, doctors fall into patterns for prescribing pharmaceuticals, particularly for those pharmaceuticals that are commonly prescribed and for those pharmaceuticals that are consistently prescribed. This invention is based, in part, on the habits of prescribing physicians with respect to various pharmaceuticals and improving the dispensing of various pharmaceuticals so that the doctors' prescriptions are accurately dispensed while at the same time reducing the burden on the pharmacists that dispense the various pharmaceuticals.

As used herein, the word “precisely” means “no more and no less then” the quantitative value following the word “precisely”. For example, in the phrase “precisely 30 unit dose compartments” the word “precisely” relates to the number of compartments but does not refer to any other characteristic of the unit dose compartment. Similarly, the word “precisely” in the phrase “precisely one unit dose” relates to the number of unit doses.

As used herein, the term “unit dose” refers to one or more pharmaceuticals to be taken generally at the same time and/or at the same time. In some circumstances a unit dose will comprise a single active pharmaceutical ingredient, in the form of, for example and without limitation, a solid, a pill, a tablet, a capsule, a powder, a gel, a liquid, or other known pharmaceutical forms and formulations. In other circumstances, a unit dose may comprise more than one active pharmaceutical ingredient. When a unit dose comprises more than one active pharmaceutical ingredient, the unit dose may be in the form of, for example and without limitation, a single solid, pill, tablet, capsule, powder, gel, liquid or other known pharmaceutical form and formulation or multiple solids, pills, tablets, capsules, powders, gels, liquids or other known pharmaceutical forms and formulations and/or combinations thereof. Such single ‘multi-pharmaceutical’ unit doses are often associated with combination therapy and/or conditions where multiple pharmaceuticals are prescribed in the same prescription and at least a portion of the pharmaceuticals are to be taken generally at the same time and/or at the same time. A single portion of the pharmaceuticals that are to be taken generally at the same time and/or at the same time falls within the term “unit dose” as used herein.

Illustrative embodiments of this invention provide a method of packaging one or more pharmaceuticals comprising separating a plurality of unit doses into a plurality of unit dose compartments, wherein each unit dose compartment of the plurality of unit dose compartments holds only one unit dose of the plurality of unit doses, each unit dose having the same strength, wherein the plurality of unit doses is precisely a quantity equal to a most commonly prescribed number of unit doses in individual prescriptions for the unit dose, or a multiple thereof.

Illustrative embodiments of this invention provide a method described herein wherein the plurality of unit doses is precisely the quantity equal to the most commonly prescribed number of unit doses in individual prescriptions for the pharmaceutical.

Illustrative embodiments of this invention provide a method described herein further comprising labeling each unit dose compartment.

Illustrative embodiments of this invention provide a method described herein wherein each unit dose compartment is a blister cavity.

Illustrative embodiments of this invention provide a method described herein further comprising obtaining quantitative prescribing information for the one or more pharmaceuticals prior to separating the plurality of doses.

Illustrative embodiments of this invention provide a method of designing packaging for one or more pharmaceuticals comprising obtaining quantitative prescribing information for the one or more pharmaceuticals; and preparing a plurality of unit dose compartments operable to hold only one unit dose and the plurality of unit dose compartments is precisely a quantity equal to a most commonly prescribed number of unit doses in individual prescriptions for the unit dose, or a multiple thereof.

Illustrative embodiments of this invention provide a method described herein wherein the quantitative prescribing information is based on the same one or more pharmaceuticals that comprise each unit dose.

Illustrative embodiments of this invention provide a method described herein wherein the quantitative prescribing information is based on one or more pharmaceuticals that are comparable to each unit dose.

Illustrative embodiments of this invention provide a kit comprising a plurality of unit doses in a plurality of unit dose compartments, wherein each unit dose compartment of the plurality of unit dose compartments holds only one unit dose of the plurality of unit doses, each unit dose having the same strength, wherein the plurality of unit doses is precisely a quantity equal to a most commonly prescribed number of unit doses in individual prescriptions for the unit dose, or a multiple thereof.

Illustrative embodiments of this invention provide a kit described herein wherein the plurality of unit doses is precisely the quantity equal to the most commonly prescribed number of unit doses in individual prescriptions for the pharmaceutical.

Illustrative embodiments of this invention provide a kit described herein wherein each unit dose compartment further comprises a label.

Illustrative embodiments of this invention provide a kit described herein wherein each unit dose compartment is a blister cavity.

Illustrative embodiments of this invention provide a kit comprising precisely 30 unit dose compartments, each unit dose compartment comprising precisely one unit dose of atorvastatin having a strength selected from the group consisting of 10 mg, 20 mg and 40 mg wherein each unit dose in each of the 30 unit dose compartments has the same strength.

Illustrative embodiments of this invention provide a kit described herein wherein the precisely 30 unit dose compartments are interspaced by perforations.

Illustrative embodiments of this invention provide a kit described herein wherein the precisely 30 unit dose compartments are blister cavities.

Illustrative embodiments of this invention provide a kit described herein wherein the precisely 30 unit dose compartments comprise three sheets, each sheet having 10 unit dose compartments.

Illustrative embodiments of this invention provide a kit described herein wherein the 10 unit dose compartments on each sheet are interspaced by perforations.

Illustrative embodiments of this invention provide a kit described herein wherein the 10 unit dose compartments on each sheet are blister cavities.

Illustrative embodiments of this invention provide a kit described herein wherein the strength of each unit dose of atorvastatin is 10 mg.

Illustrative embodiments of this invention provide a kit described herein wherein the strength of each unit dose of atorvastatin is 20 mg.

Illustrative embodiments of this invention provide a kit described herein wherein the strength of each unit dose of atorvastatin is 40 mg.

Illustrative embodiments of this invention provide a method of packaging atorvastatin comprising separating 30 unit doses of atorvastatin into 30 unit dose compartments, wherein each one of the 30 unit dose compartments each holds only one of the 30 unit doses of atorvastatin, each unit dose of atorvastatin having a strength selected from the group consisting of 10 mg, 20 mg and 40 mg and each unit dose of atorvastatin dose having the same strength.

Illustrative embodiments of this invention provide a method described herein wherein the 30 unit dose compartments are 30 blister cavities.

Illustrative embodiments of this invention provide a method described herein wherein the 30 blister cavities are further packaged inside a box.

Illustrative embodiments of this invention provide a method described herein wherein the strength of each unit dose of atorvastatin is 10 mg.

Illustrative embodiments of this invention provide a method described herein wherein the strength of each unit dose of atorvastatin is 20 mg.

Illustrative embodiments of this invention provide a method described herein wherein the strength of each unit dose of atorvastatin is 40 mg.

Illustrative embodiments of this invention provide a kit comprising precisely eight unit dose compartments, each unit dose compartment comprising precisely one unit dose of 500 mg valacyclovir.

Illustrative embodiments of this invention provide a kit described herein wherein the precisely eight unit dose compartments are interspaced by perforations.

Illustrative embodiments of this invention provide a kit described herein wherein the precisely eight unit dose compartments comprise four sheets, each sheet having two unit dose compartments.

Illustrative embodiments of this invention provide a kit described herein wherein the two unit dose compartments on each sheet are interspaced by perforations.

Illustrative embodiments of this invention provide a kit described herein wherein the unit dose compartments are blister cavities.

Illustrative embodiments of this invention provide a kit comprising precisely four unit dose compartments, each unit dose compartment comprising precisely one unit dose of valacyclovir having a strength selected from the group consisting of 500 mg and 1000 mg wherein each unit dose in each of the eight unit dose compartments has the same strength.

Illustrative embodiments of this invention provide a kit described herein wherein the precisely four unit dose compartments are interspaced by perforations.

Illustrative embodiments of this invention provide a kit described herein wherein the precisely four unit dose compartments comprise two sheets, each sheet having two unit dose compartments.

Illustrative embodiments of this invention provide a kit described herein wherein the two unit dose compartments on each sheet are interspaced by perforations.

Illustrative embodiments of this invention provide a kit described herein wherein the unit dose compartments are blister cavities.

Illustrative embodiments of this invention provide a kit described herein wherein the strength of each unit dose of valacyclovir is 500 mg.

Illustrative embodiments of this invention provide a kit described herein wherein the strength of each unit dose of valacyclovir is 1000 mg.

Illustrative embodiments of this invention provide a method of packaging valacyclovir comprising separating eight unit doses of 500 mg valacyclovir into eight unit dose compartments wherein each one of the eight unit dose compartments each holds only one of the eight unit doses of 500 mg valacyclovir.

Illustrative embodiments of this invention provide a method described herein wherein the eight unit dose compartments are eight blister cavities.

Illustrative embodiments of this invention provide a method described herein wherein the eight blister cavities are further packaged inside a box.

Illustrative embodiments of this invention provide a method of packaging valacyclovir comprising separating four unit doses of valacyclovir into four unit dose compartments wherein each one of the four unit dose compartments each holds only one of the four unit doses of valacyclovir, each unit dose of valacyclovir having a strength selected from the group consisting of 500 mg and 1000 mg and each unit of valacyclovir dose having the same strength.

Illustrative embodiments of this invention provide a method described herein wherein the four unit dose compartments are four blister cavities.

Illustrative embodiments of this invention provide a method described herein wherein the four blister cavities are further packaged inside a box.

Illustrative embodiments of this invention provide a method described herein wherein the strength of each unit dose of valacyclovir is 500 mg.

Illustrative embodiments of this invention provide a method described herein wherein the strength of each unit dose of valacyclovir is 1000 mg.

Other aspects and features of the present invention will become apparent to those ordinarily skilled in the art upon review of the following description of specific embodiments of the invention.

### DETAILED DESCRIPTION

Pharmaceutical packaging is available in many different varieties. Each variety of packaging provides different advantages and disadvantages. The present invention is based, in part, on the properties of various pharmaceuticals, the prescribing practices of physicians for such various pharmaceuticals and relating such properties and practices to the advantages and disadvantages of a particular variety of packaging.

Blister sheets are generally known arrangements for dispensing medicaments in solid form or in powder form. A blister sheet comprises a blister cavity in which a unit dose lies and such a cavity is enclosed by a blister base on one side and a material defining the blister cavity on the remaining sides. Blister sheets are often made up of several layers of different materials. For example, a four layer blister sheet may comprise two base panels of a suitable material such as plastic and/or board and/or paper. Between these two base panels two additional layers of material are placed, one of which defines the blister cavity in which a unit dose lies and the other forms an easily pierced and/or removed blister base.

A two layer blister sheet structure can also be used in which the blister cavities are defined by a layer of material and the layer of material is then coated with a material that provides the blister base. The material that provides the blister base is often, but need not be, a lidding foil and is typically made of an easily pierced material. Another common material for use as a blister base is board, such as cardboard or paper, and often this sort of blister base comprises perforations to allow for easy separation of the blister base from the rest of the blister sheet.

Some of the materials known to the skilled person and used for producing commercially available blister sheets are, for example, cardboard, paper, other cellulosic products, laminated aluminum foils, PVC (polyvinyl chloride) or PVdC (polyvinylidene chloride) foils or combinations of these, or PP (polypropylene) foils or combinations or laminates of such materials.

Blister packs may be operated by breaking the material of the blister base and then removing the unit dose from the blister cavity through the break in the material of the blister base. The break in the blister base is often made by application of pressure at the location where the unit dose is in contact with the blister base. This is often achieved by the user applying pressure to the material defining the blister cavity.

Blister sheets are often further packaged in a rectangular box which can be opened and closed at opposite short narrow ends. Opening and closing is done by pulling out or pushing in a tuck-in tab. The tuck-in tabs are often produced in one piece with the rest of the box from a common blank. The box can be printed with patient information, such as name of medicament, logo(s), indication(s), strength, pack size, expiry date, etc., and it is filled with the blister sheet or blister sheets and often an insert containing detailed information for the patient.

In addition to being used in combination with other sorts of packaging, such as boxes, blister sheets may be also be integral with other packaging or may be the only type of packaging used in embodiments of the present invention. In some illustrative embodiments of the present invention, blister sheets may comprise one or more folds such that a book-like package is formed. In other cases, the one or more folds may form a tri-fold package. In other cases still, the one or more folds may form a leaflet and/or pamphlet-like package in which several surfaces of the blister sheet may be used. In some cases more than one surface of the blister sheet may comprise exit points for a unit dose. In some cases, one or more surfaces of the blister sheet may comprise no exit points for a unit dose and such surfaces may be used for printed information. In some cases a blister sheet surface will comprise an exit point for unit dose as well as printed information.

The present invention is based, in part, on the results from a study of pharmacists' dispensing habits. The study of pharmacists revealed the following analogies regarding blister packaging. Dispensing medication in blister packaging is . . .
Like taking a break because there is no preparation necessary
Like using saran wrap because it is protected, well ordered and preserved
Like driving a Ferrari because it is pre-counted and fast
Like dressing a six year old because sometimes it works as it should and other times it is much more work than it should be
Like doing arts and crafts because [one needs] to cut and trim the packaging according to the quantity of the prescription [(if the prescription is for less than the number of blisters on the sheet)].

Based in part on the foregoing, the present inventors developed a kit comprising a plurality of unit doses in a plurality of unit dose compartments, wherein each unit dose compartment holds only one unit dose and the number of individual unit doses in the plurality of unit doses is precisely a quantity equal to a most commonly prescribed number of unit doses in individual prescriptions for the unit dose or a multiple thereof. Such a kit may provide pharmacists with the benefits of individual unit dose packaging, such as blister packaging, while at the same time reducing some of the deficiencies of such packaging.

In one embodiment of the present invention, there is provided a method of packaging one or more pharmaceuticals. The method comprises separating a plurality of unit doses into a plurality of unit dose compartments. Each unit dose compartment of the plurality of unit dose compartments holds precisely one unit dose of the plurality of unit doses. Each unit dose in a particular package has the same strength. The plurality of unit doses is precisely a quantity equal to a most commonly prescribed number of unit doses in individual prescriptions for the unit dose, or alternatively may be a multiple thereof. Similarly, it is commonly the case that the plurality of unit dose compartments is also precisely a quantity equal to a most commonly prescribed number of unit doses in individual prescriptions for the unit dose, or alternatively may be a multiple thereof.

The strength of a unit dose may be determined by measuring an amount of active pharmaceutical ingredient(s) in the dose. The quantity equal to the most commonly prescribed number of unit doses in individual prescriptions for the unit dose may be determined by conducting at least one analysis of the quantitative prescribing habits for the particular unit dose of a representative sample of doctors, by conducting an analysis of the number and nature of insurance claims for a particular unit dose, or by any means known in the art for identifying the quantitative prescribing habits of doctors.

Analyses may be conducted in particular geographic locations. The geographic location may be selected based on the prevalence of the use of a particular unit dose, the particular legal boundaries associated with prescribing habits or a combination thereof. For example, an analysis of a continent, such as Europe may be conducted. Alternatively, an analysis of a subset of the countries within a continent may be conducted, such as an analysis including France, Germany, Switzerland and the United Kingdom. Alternatively an analysis of a country, such as Canada or the United States may be conducted. Further still an analysis of a sub set of states or provinces of a particular country may be conducted. Alternatively an analysis of an individual province or state of a particular country may be conducted. In some cases it may be beneficial to conduct an analysis of a subset of cities and/or towns in a particular continent, country, province and/or state. Analyses may be broad-ranging so that many geographic areas are covered and the single broad ranging analysis may be statistically organized so that different geographic regions may be identified based on prescribing habits that may be recognized by review of the analysis.

Analyses may, for example, be conducted at any time after a unit dose has been sold in the market. Often an analysis is not conducted until the unit dose has been sold in the market for at least a period of 1 month. In other embodiments, the analysis is not conducted until the unit dose has been sold in the market for at least 1 year. In other embodiments, the analysis is not conducted until the unit dose has been sold in the market for at least 2 years. In other embodiments, the analysis is not conducted until the unit dose has been sold in the market for at least 3 years. In other embodiments, the analysis is not conducted until the unit dose has been sold in the market for at least 4 years. In other embodiments, the analysis is not conducted until the unit dose has been sold in the market for at least 5 years. In other embodiments, the analysis is not conducted until the unit dose has been sold in the market for at least 6 years. In other embodiments, the analysis is not conducted until the unit dose has been sold in the market for at least 7 years. In other embodiments, the analysis is not conducted until the unit dose has been sold in the market for at least 8 years. In other embodiments, the analysis is not conducted until the unit dose has been sold in the market for at least 9 years. In other embodiments, the analysis is not conducted until the unit dose has been sold in the market for at least 10 years. It is often beneficial to conduct an additional analysis if the unit dose has been approved for a new indication.

In some cases the consideration of the geographic location and the length of time the unit dose has been sold in the market may go hand in hand. For example, the unit dose may not be approved for use in one geographic location until long after it has been approved for use in another geographic location. In such a case, it may be beneficial to conduct an analysis which allows for statistical analysis of both the geographic location of the prescription for the unit dose and the length of time that the unit dose has been sold in the market in the particular geographic location.

It is also possible to perform an analysis on one or more comparable pharmaceutical products. For example, if the unit dose of interest is to be prescribed for an indication for which there is a comparable pharmaceutical prescribed and the dosing regimes for the comparable pharmaceutical and the unit dose of interest are likely to be and/or are similar, then an analysis of the comparable pharmaceutical may be undertaken. Alternatively, if there are two comparable pharmaceutical products, then a single analysis, or two separate analyses, may be undertaken, and the results for both comparable pharmaceutical products may be used. Such analyses may be conducted before the unit dose of interest is sold in the market. The results of such analyses may be used in the preparation of pharmaceutical packaging according to the present invention. The comparable pharmaceutical may be, for example, a pharmaceutical which is the first market incident of a particular active pharmaceutical ingredient, a generic equivalent, and/or a different active pharmaceutical ingredient prescribed for the same indication or a combination thereof.

Some pharmaceuticals and/or unit doses that may be suited to packaging according to the present invention are included, without limitation in the following list. The following list contains the names of active pharmaceutical ingredients, product trade names and/or combinations thereof: abacavir; abacavir+lamivudine; abacavir sulfate+lamivudine; abacavir sulfate+lamivudine+zidovudine; abacavir sulphate; abatacept; abbokinase; abciximab; abelcet; abilify; abreva; acamprosate calcium; acarbose; accolate; accuneb; accupril; accuretic; accutane; accutrim; accuzyme; ace inhibitors; ace inhibitors+thiazide; acebutolol; aceon; acephen; acetadote; acetaminophen; acetaminophen+caffeine+codeine; acetaminophen+phenyltoloxamine; acetaminophen+tramadol; acetaminophen+isometheptene+dichloralphenazone; acetasol; acetasol hc; acetazolamide; acetic acid; acetic acid+aluminum acetate; acetic acid+desonide; acetic acid+hydrocortisone; acetic acid+oxyquinoline+ricinoleic acid; acetohexamide; acetohydroxamic acid; acetylcysteine; acetylsalicylic acid; achromycin; achromycin v; acid reducer; aci-jel; aciphex; acitretin; actigall; actimmune; actinex; actiq; activase; activella; actonel; actoplus met; actos; acular; acular ls; acyclovir; adagen; adalat; adalat cc; adalimumab; adapalene; adapin; adderall; adderall xr; adefovir dipivoxil; adenosine; adgan; adipex-p; adrenalin; adrenocot; adriamycin; adrucil; adsorbonac; advair diskus; advicor; advil; aerobid; afrin; afrinol; agenerase; aggrastat; aggrenox; agrylin; a-hydrocort; airet; akarpine; ak-cide; ak-con-a; akne-mycin; ak-spore hc; ak-t-caine pf; ak-tracin; alamast; alatrofloxacin; alavert; albaform hc; albalon; albatussin; albatussin la; albendazole; albenza; albumin; albuminar; albuterol; albuterol+ipratropium; albuterol pediatric; albuterol sulfate; albuterol sulfate+ipratropium; alcaine; alcohol acetone; aldactone; aldara; aldesleukin; aldomet; alefacept; alemtuzumab; alendronate; alendronate+cholecalciferol; alendronate sodium; alendronate sodium+vitamin d3; alert; aleve; alfenta; alfentanil; alferon-n; alfuzosin; alfuzosin hydrochloride; alglucerase; alinia; aliskiren hemifumarate; alitretinoin; alkeran; allegra; allegra-d; allerhist-1; allopurinol; allopurinol sodium; almotriptan; almotriptan maleate; alocril; alomide; aloprim; alosetron; aloxi; alpha-1-proteinase inhibitor; alphagan; alphagan p; alphanate; alphanine sd; alprazolam; alprazolam intensol; alprostadil; alrex; altace; altaseptic; alteplase; alternagel; altinac; altocor; altoprev; altretamine; alu-cap; aluminum carbonate; aluminum chloride; aluminum hydroxide; aluminum+magnesium hydroxide; aluminum+calcium+magnesium; alupent; alu-tab; alvimopan; amantadine; amaryl; ambenonium; ambien; ambien cr; ambisome; ambrisentan; amen; amerge; americaine; amevive; amicar; amidal; amidrine; amifostine; amikacin; amikin; amiloride; amino acid-electrolytes-glycerin; amino acids; aminocaproic acid; amino-cerv; aminoglutethimide; aminolevulinic acid; aminophylline; aminosyn; amiodarone; amiodarone hydrochloride; amitriptyline; amitriptyline w+perphenazine; amitriptyline+chlordiazepoxide; amlexanox-mucous membrane; amlodipine; amlodipine+atorvastatin; amlodipine+benazepril; amlodipine base; amlodipine besylate; amlodipine besylate+atorvastatin; amlodipine maleate; amlodipine w+benazepril; ammonium biphos-sodium biphos-sodium acid pyropho; ammonium cl-phenylephrine-hydrocodone-pyrilamine; ammonium lactate; amnesteem; amoply; amoxicillin; amoxicillin+clavulanate; amoxicillin+clavulanic acid; amoxil; amphetamine+dextroamphetamine; amphocin; amphojel; amphotec; amphotericin; ampicillin; ampicillin+sulbactam; amprenavir; amprenavir+vitamin e; amprenavir+vitamin e+propylene glycol; anabar; anabolic steroids; anadrol; anafranil; anagrelide; ana-guard; anakinra; ana-kit; analgesics+antipyretics; anaprox; anaspaz; anastrozole; ancobon; androderm; androgel; android; anexsia; angiomax; angiotensin blockers; angiotensin blockers w+hydrochlorothiazide; angiotensin converting enzyme inhibitor; angiotensin ii receptor blockers; anidulafungin; anileridine; anisindione; anistreplase; ansaid; antabuse; anthralin; antibacterials; antibiotic+anesthetic+steroid; antibiotic combinations+hydrocortisone; antibiotic+steroid; antibiotic+anesthetic+steroid; anticholinergics-antispasmodics; antidiabetics; antidyskinetics; antifungals; antihemophilic factor; antihistamines; antihistamines+decongestants; anti-inhibitor coagulant complex; antipyrine+benzocaine; antivert; antrizine; anturane; anucort-hc; anusert he-1; anusol-hc; anzemet; apap; aphrodyne; aphthalsol; appetite suppressants; apraclonidine; aprepitant; apresazide; apresoline; apri; aqua-mephyton; aquasol a; aquasol e; aralen; aranesp; arava; arb; ardeparin; aredia; argatroban; aricept; aricept odt; arimidex; aripiprazole; arixtra; arm-a-med; arnica; aromasin; aromatic ammonia; arsenic trioxide; arthrotec; asacol; ascorbate calcium; ascorbic acid; ascriptin; ashwagandha; asmanex; asparaginase; a-spas-s+l; aspergum; aspirin; aspirin+antiplatelet medications; aspirin chewable; aspirin gum; aspirin therapy; aspirin+meprobamate; aspirin+dipyridamole er; astelin; astemizole; atacand; atamet; atarax; atarin; atazanavir; atazanavir sulfate; atenolol; atenolol+chlorthalidone; atgam; ativan; atomoxetine; atomoxetine hydrochloride; atorvastatin; atorvastatin calcium; atorvastatin calcium+amlodipine besylate; atovaquone; atovaquone+proguanil hydrochloride; atovaquone+proguanil; atralin; atromid-s; atropine; atropine sulfate; atropisol; atrovent; attapulgite; atuss ex; atuss-12 dx; augmentin; augmentin es-600; augmentin xr; auralgan; auranofin; auro; aurodex; aurolate; aurothioglucose; aurothiomalate; auroto; autoplex t; avage; avalide; avandamet; avandia; avapro; avar; avastin; avc; avelox; avelox iv; aventyl; aviane; avinza; avita; avodart; avonex; axert; axid; axotal; aygestin; azacitidine; azactam; azathioprine; azelaic acid; azelastine; azelastine hydrochloride; azelex; azithromycin; azmacort; azo-gantanol; azo-gantrisin; azopt; aztreonam; azulfidine; b+o supprettes; baby gas; baciguent; bacitracin; baclofen; bacticin; bactrim; bactroban; balsa-derm; balsalazide; balsalazide disodium; barbiturates; basaljel; basiliximab; baycol; bearberry; bebulin vh immuno; becaplermin; beclomethasone; beclomethasone dipropionate; beconase; beepen vk; belladonna alkaloids+barbiturates; belladonna+opium suppository; bellamine-s; bellaspas; bellergal-s; benadryl; benazepril; benazepril+hydrochlorothiazide; benefix; benemid; bengay; benicar; benicar hct; benoquin; benoxyl; benserazide+levodopa; bentyl; benylin; benzac ac; benzac w; benzaclin; benzagel; benzamycin; benzocaine; benzocaine-antipyrine-phenylephrine; benzodiazepines; benzoin; benzonatate; benzoyl peroxide; benzoyl peroxide+erythromycin; benzphetamine; benztropine mesylate; benzydamine; benzyl alcohol+pramoxine; bepridil; beta blockers; beta-blockers w+thiazide diuretics; betachron; betahistine; betaine; betamethasone; betamethasone dipropionate; betamethasone sodium; betamethasone-calcipotriol; betamethasone-salicylic acid; betamine; betapace; betaseron; betaxolol; betaxolol hydrochloride; bethanechol; bethaprim; betoptic; betoptic.s; bevacizumab; bexarotene; bextra; bezafibrate; biaxin; biaxin xl; bicalutamide; bicillin cr; bicillin l-a; bicitra; bicnu; bifidobacterium bifidum; bilberry fruit; bilberry leaf; bile acid sequestrants; biltricide; bimatoprost; bioclate; bio-statin; birch leaf; bisacodyl; bismuth subsalicylate; bisoprolol; bisoprolol+hydrochlorothiazide; bisoprolol fumarate; bitolterol; bitolterol mesylate; bivalirudin; black cohosh; blenoxane; bleomycin; blephamide; blephamide s.o.p.; blocadren; blue green algae; boldo; bonine; boniva; borage seed oil; bortezomib; bortezomib mannitol boronic ester; bosentan; botox; botox cosmetic; botulinum toxin; botulinum toxin type a; bravelle; brethine; bretylium tosylate; brevibloc; brevital; brevoxyl; bricanyl; brimonidine; brimonidine tartrate; brimonidine tartrate+timolol; brinzolamide; bromazepam; bromfenac; bromocriptine; bronchodilator; broncholate; broncomar gg; broncomar-1; brondelate; bronkaid; bronkometer; bronkosol; budeprion sr; budeprion xl; budesonide; budesonide+formoterol; bufferin; bulk-producing laxatives; bumetanide; bumex; buminate; buphenyl; bupivicaine; buprenorphine; buprenorphine hydrochloride; buprenorphine-naloxone; bupropion; bupropion hydrochloride; burrow's; buscopan; buspar; buspirone; busulfan; busulfex; butalbital+acetaminophen+caffeine; butalbital w+acetaminophen; butalbital+aspirin; butalbital+aspirin+caffeine; butazolidine; butenafine; butorphanol; butorphanol tartrate; byetta; cabergoline; caduet; cafatine pb; cafcit; cafergot; caffedrine; caffeine; caffeine citrate; caladryl; caladryl clear; calahist clear; calamine; calan; calan sr; calcidrine; calciferol; calcijex; calcimar; calcipotriene; calcipotriene+bethamethasone; calcipotriol; calcitonin; calcitonin salmon; calcitriol; calcium acetate; calcium carbonate; calcium carbonate+vitamin b12+folic acid+vitamin; calcium carbonate antacid; calcium channel blockers; calcium supplement+vitamin d; calcium supplements; calderol; caltrate 600; caltrate 600 plus; caltrate 600 plus d; cama; campath; camphorated opium; campral; camptosar; cancidas; candesartan; candesartan cilexetil; candesartan cilexetil+hydrochlorothiazide; cantil; capastat sulfate; capecitabine; capitrol; capoten; capozide; capreomycin; capsagel; capsaicin; capsicum; captopril; captopril+hydrochlorothiazide; captopril+thiazide; carac; carafate; caramiphen+phenylpropanolamine; carbachol; carbamazepine; carbamide peroxide; carbatrol; carbenicillin+bacampicillin; carbetapentane-chlorpheniramine; carbetapentane-chlorpheniramine-phenylephrine; carbetapentane-decongestant-antihistamine; carbetapentane-phenylephrine-pyrilamine; carbex; carbidopa; carbidopa+entacapone; carbidopa+levodopa; carbidopa-levodopa-entacapone; carboplatin; carboprost tromethamine; carboptic; cardene; cardene sr; cardizem; cardizem cd; cardizem la; cardizem sr; cardura; carisoprodol; carisoprodol w+aspirin; carmex; carmol; carmustine; carmustine-polifeprosan 20; carnitor; carteolol; carvedilol; carvedilol phosphate; casodex; caspofungin; cataflam; catapres; catapres-tts; cathflo activase; catulac; caverject; ccbs; ceclor; cecon; cedax; ceenu; cefaclor; cefaclor sa; cefadroxil; cefazolin; cefdinir; cefditoren; cefepime; cefixime; cefotaxime; cefoxitin; cefpodoxime proxetil; cefprozil; ceftazidime; ceftibuten; ceftin; ceftriaxone; cefuroxime axetil; cefzil; celebrex; celecoxib; celexa; cellcept; celontin; cenestin; cephalexin; cephalosporins; cephradine; cephulac; cerebyx; ceredase; cerezyme; cerivastatin; ceron-dm; cerubidine; cerumenex; cervidil; cetapred; cetazone t; cetirizine; cetirizine syrup; cetrorelix; cetrotide; cevimeline; cevimeline hydrochloride; chamomile; chantix; chaste tree; chemet; cheracol; cheracol d; chibroxin; children's advil; children's aspirin; children's motrin; children's mylanta; chloral hydrate; chlorambucil; chloramphenicol; chloramphenicol w+hydrocortisone; chloramphenicol-fibrinolysin-dnase; chloramphenicol-hydrocortisone; chloramphenicol-polymyxin-hydrocortisone; chlordiazepoxide; chlordiazepoxide hydrochloride+clidinium bromide; chlordiazepoxide+clidinium; chlorhexidine; chloroquine; chloroxine; chlorpheniramine+hydrocodone; chlorpheniramine+pseudoephedrine; chlorpromazine; chlorpromazine spansules; chlorpropamide; chlorprothixene; chlorthalidone; chlorthalidone+atenolol; chlor-trimeton; chlorzoxazone; chlorzoxazone+acetaminophen; cholac; cholestyramine; cholestyramine+colestipol; choline magnesium salicylate; choline salicylate-magnesium salicylate; chorex; choriogonadotropin alfa; chromagen; chromagen forte; chronulac; chymodiactin; chymopapain; cialis; ciclesonide; ciclopirox; cidofovir; cilazapril; cilazapril+hydrochlorothiazide; cilostazol; ciloxan; cimetidine; cinacalcet; cinobac; cinoxacin; cipro; cipro hc; cipro xr; cipro xr; ciprodex; ciprofloxacin; ciprofloxacin+dexamethasone; ciprofloxacin+hydrocortisone; ciprofloxacin hydrochloride+dexamethasone; cisapride; cisplatin; citalopram; citracal+d; citric acid+potassium-sodium citrates; citroma; cladribine; claravis; clarif; clarinex; clarinex reditabs; claripel; clarithromycin; claritin; claritin reditabs; claritin-d; clear eyes; clemastine; cleocin; cleocin pediatric; cleocin t; clidinium bromide+chlordiazepoxide hydrochloride; climara; climara pro; clinda-derm; clindamycin; clindamycin+benzoyl peroxide; clindamycin phosphate; clinoril; clioquinol; clioquinol+hydrocortisone; clobazam; clobetasol; clobex; clofazimine; clofibrate; clomid; clomiphene; clomipramine; clonazepam; clonidine; clonidine w+chlorthalidone; clopidogrel; clopidogrel bisulfate; clorazepate; clotrimazole; clotrimazole+betamethasone dipropionate; clotrimazole lozenge mucous membrane; clotrimazole+betamethasone; clozapine; clozaril; cmv immune globulin; coal tar; cobal; cocaine hydrochloride; codamine; codeine; codeine+acetaminophen; codeine+asa+caffeine+meprobamate; codeine+asa+phenobarbital; codeine+aspirin+caffeine+butalbital; codeine+carisoprodol+aspirin; codeine+chlorpheniramine+iodinated glycerol-or; codeine+chlorzoxazone+apap; codeine+calcium iodide syrup; codeine phosphate; codeine+iodinated glycerol; coenzyme q10; cogentin; cognex; colace; colazal; col-benemid; colchicine; colchicine+probenecid; colesevelam; colestid; colestipol; colistimethate; colistin sodium methate; colistin sulfate; collagenase oint; col-probenecid; coly-mycin m; coly-mycin s; colyte; combination antibiotic; combination antibiotic+steroid; combination antibiotic+steroid; combipatch; combipres; combivent; combivir; comfrey; compazine; comtan; comvax; concerta; condylox; conjugated estrogens; conjugated estrogens+medroxyprogesterone; constilac; constulose; contrin; copaxone; copegus; cordarone; cordran; coreg; coreg cr; corgard; cormax; coromega; corque; cortane-b; cort-dome; cortef; cortenema; cortic; corticosteroids; cortifoam; cortisporin; cortisporin-tc; cortrosyn; cosmegen; cosopt; cosyntropin; cotazym-s; cotrim; cotuss-v; coumadin; coumadin tabs; coumarin-type drugs; covera-hs; covera-hs; cox-2 inhibitors; cozaar; cpc-cort-d; cranberry; creon; crestor; crinone; crixivan; crixivan; crolom; cromolyn; crotamiton; crystodigin; cubicin; cuprimine; cutivate; cyanocobalamin; cyanoject; cyclandelate; cyclizine-ergotamine-caffeine; cyclobenzaprine; cyclogyl; cyclopentolate hydrochloride; cyclophosphamide; cycloserine; cyclospasmol; cyclosporine; cyclosporine microemulsion; cyclosporine-iv; cycrin; cyklokapron; cylate; cylert; cymbalta; cyomin; cyproheptadine; cyproterone; cyproterone acetate; cystadane; cystagon; cysteamine; cystospaz; cytadren; cytarabine; cytarabine liposome; cytogam; cytomel; cytosar-u; cytotec; cytovene; cytoxan; cytra-2; d.h.e.45; dacarbazine; daclizumab; dactinomycin; dailyhist-1; dalgan; dalteparin; damiana; danaparoid; danazol; dandelion; dandrex; danocrine; dantrium; dantrolene; dapiprazole; dapsone; daptacel; daptomycin; daraprim; darbepoetin alfa; darifenacin bromide; darvocet; darvocet a500; darvocet-n; darvon; darvon cmpd-65; darvon-n; das; dasantib; daunorubicin; daunorubicin citrate liposome; daunoxome; daypro; debacterol; debrox; decadron; decadron phosphate; decaject-10; decitabine; declomycin; deco-d; deconamine; deconamine cx; decongestant; decongestant+antihistamine+anticholinergic; decongestant+narcotic antitussive; decongestant-narcotic antitussive-antihistamine; decongestant-narcotic antitussive-apap-antihist; deconsal; deferiprone; deferoxamine; definity; delatestryl; delavirdine; delestrogen; delone; deltasone; demadex; demecarium bromide; demeclocycline; demerol; denavir; denileukin diftitox; denorex; deoxyribonuclease; depacon; depakene; depakote; depakote er; depakote sprinkle; depen; dep-gynogen; depocyt; depo-estradiol; depogen; depo-medrol; deponit; depo-provera; derma-smoothe+fs; desferal; desipramine; desitin; desloratadine; desmopressin; desmopressin acetate; desoximetasone; desoxyn; desquam; desyrel; detrol; detrol la; detue; devil's claw; dexamethasone; dexatrim; dexedrine; dexfenfluramine; dexferrum; dexmethylphenidate; dexpanthenol+choline; dexrazoxane; dextroamphetamine; dextroamphetamine+amphetamine; dextromethorphan; dextromethorphan+potassium guaiacolsulfonate-o; dextromethorphan+decongestant; dextromethorphan+iodinated glycerol; dextromethorphan+decongestant+antihistamine; dextrose; dextrose+sodium chloride; dextrostat; dezocine; dha+epa-policosanol-vitamin b12+b6-folic acid; dht; diabeta; diabinese; dialose; dialume; diamox; diaphragm; diasorb; diastat; diatrizoate meglumine+diatrizoate sodium; diazepam; diazepam emulsion; diazoxide; dibenzyline; dicarbosil; diclofenac; diclofenac+misoprostol; diclofenac potassium; diclofenac sodium; diclofenac w+misoprostol; dicyclomine; didanosine; didrex; didronel; dienestrol; difenoxin w+atropine sulfate; differin; diflucan; diflunisal; di-gel; digestive aids; digestive enzymes; digitalis medicine; digitek; digoxin; dihydroergotamine; dihydrotachysterol; dilacor; dilacor xr; dilantin; dilantin kapseals; dilaudid; dilor; dilor-g; diltiazem; diltiazem hydrochloride; dimenhydrinate-ergotamine-caffeine; dimethyl sulfoxide; dinoprostone; diovan; diovan hct; dipentum; diphenhydramine; diphenidol hcl; diphenoxylate+atropine; diphtheria+tetanus toxoids combined; diprolene; diprosone; dipyridamole; dipyridamole+acetylsalicylic acid; dirithromycin-ec; disalcid; disalcid; disopyramide; dispermox; disulfiram; disurex d+s; ditropan; ditropan xl; diulo; divalproex; divalproex sodium; doan's; dobutamine; dobutrex; docetaxel; docosanol; docusate; dofetilide; dolasetron; dolasetron mesylate; dolobid; dologesic; dolophine; dolorex; dolsed; domeboro; domperidone; donepezil; donepezil hydrochloride; dong quai; donnagel; donnamar; donnatal; dopamine; dopar; dopram; dornase alpha; dorzolamide; dorzolamide hydrochloride; dorzolamide hydrochloride+timolol maleate; dostinex; dovonex; doxapram; doxazosin; doxazosin mesylate; doxepin; doxercalciferol; doxil; doxorubicin; doxorubicin hydrochloride; doxorubicin w+liposomes; doxycycline; doxylamine succinate+pyridoxine; doxylamine+pyridoxine; dramamine ii; drisdol; dronabinol; droperidol; drotrecogin alfa; drysol; dss; dtic-dome; duac; duet dha; dulcolax; duloxetine; duloxetine hydrochloride; duoneb; duphalac; duraclon; duract; duragesic; duratuss; dura-vent; duricef; dutasteride; dyazide; dynabac; dynacin; dynacirc; dyphylline; dyrenium; ear antibiotics; earsol-hc; easprin; echinacea; echothiophate iodide; ec-naprosyn; econazole; ecotrin; edecrin; ed-flex; efalizumab; efavirenz; effexor; effexor xr; eflornithine; efudex; elase; elase-chloromycetin; elavil; eldepryl; elder; eldoquin; electrolyte; electrolyte+peg; elestat; eletriptan; eletriptan bromide; elidel; eligard; elimite; elitek; elixophyllin ki; elixsure ib; ellence; elmiron; elocon; eloxatin; elspar; emadine; emcyt; emedastine; emend; emgel; eminase; emla; emollients; empirin; emtricitabine; emtricitabine-tenofovir; emtriva; e-mycin; enablex; enalapril; enalapril+hydrochlorothiazide; enalapril maleate; enalapril maleate+hydrochlorothiazide; enalaprilat; enalapril-diltiazem; enalapril-felodipine; enbrel; encainide; endal; endep; endocaine; endocet; enfuvirtide; enfuvirtide; engerix-b; enoxacin; enoxaparin; enoxaparin sodium; entacapone; entecavir; entex; entex la; entocort ec; entuss; enulose; enzone; ephedrine; epi e-z pen; epifoam; e-pilo-1; epinastine; epinastine hydrochloride; epinephrine; epinephrine allergy syringe; epinephrine+chlorpheniramine; epinephrine-type glaucoma eye medicine; epipen; epiquin micro; epirubicin hydrochloride; epivir; epivir hbv; eplerenone; epoetin alfa; epogen; epoprostenol; epoprostenol sodium; eptifibatide; epzicom; equagesic; equalactin; equalyte; equanil; equetro; ercaf; ergamisol; ergoloid mesylates; ergomar; ergonovine maleate; ergotamine; ergotamine+caffeine; ergotamine-belladonna-phenobarbital; ergotamine-caffeine-belladonna alk; ergotamine-caffeine-belladonna-pentobarb; erlotinib; erlotinib hydrochloride; ertapenem; eryc; erycette; erygel; eryped; ery-tab; erythrityl tetranitrate; erythrocin; erythromycin; erythromycin+benzoyl peroxide; erythromycin base; erythromycin base+erythromycin stearate; erythromycin estolate; erythromycin ethylsuccinate; erythromycin gluceptate; erythromycin lactobionate; erythromycin stearate; erythromycin+benzoyl peroxide; erythromycin+sulfisoxazole; erythromycin-tretinoin; eryzole; escitalopram; escitalopram oxalate; esclim; esgic; esimil; eskalith; esmolol; esmolol hydrochloride; esomeprazole; esomeprazole magnesium; esomeprazole sodium; esterified estrogens; esterified estrogens+methyltestosterone; estinyl; estrace; estraderm; estradiol; estradiol+ethinyl estradiol; estradiol+levonorgestrel; estradiol+medroxyprogesterone; estradiol+norethindrone; estradiol acetate; estradiol cypionate; estradiol valerate; estradiol-norethindrone acetate-patch; estramustine phosphate; estrasorb; estratab; estratest; estring; estrogel; estrogen+progestins; estrogens; estrogens conjugated; estrogens conjugated+synthetic; estrogens+chlordiazepoxide; estrogens+meprobamate; estropipate; estrostep fe; eszopiclone; etanercept; ethacrynate sodium; ethacrynic acid; ethambutol; ethatab; ethaverine; ethavex; ethchlorvynol; ethedent; ethinyl estradiol+drospirenone; ethinyl estradiol+norelgestromin patch; ethinyl estradiol-cyproterone; ethionamide; ethmozine; ethosuximide; ethotoin; ethyol; etidronate; etidronate+calcium; etidronate+calcium carbonate; etidronate disodium; etodolac; etonogestrel+ethinyl estradiol; etopophos; etoposide; etoposide phosphate; etrafon; etretinate; eulexin; eurax; evalose; evening primrose oil; everone; evista; evoxac; exelon; exemestane; exenatide; exotic-hc; expectorant-decongestant-narcotic antitussive; expectorants-narcotic antitussive-antihistamine; exsel; eye lubricant; ezetimibe; ezetimibe+simvastatin; fa-8; factive; factor ix human recombinant; factor ix human; factor viia recombinant; famciclovir; famotidine; famotidine+calcium carbonate+magnesium hydroxide; famvir; fansidar; fareston; faslodex; fastin; fat emulsions; felbamate; felbatol; feldene; felodipine; femara; femiron; femring; fenesin; fenofibrate; fenoprofen; fenoterol; fentanyl; fentanyl citrate; fentora; fenugreek; feosol; fergon plus; fer-in-sol; fero-folic; fero-grad; ferragen; ferralet plus; ferrlecit; fertinex; fever reducer; feverall; feverfew; fexofenadine; fexofenadine+pseudoephedrine; fibrates; fibrinolysin w+dnase; filgrastim; finacea; finasteride; fioricet; fiorinal; fiorinal w+codeine #3; fiormor; flagyl; flagyl er; flatulex; flavoxate; flaxseed; flecainide; flecainide acetate; fleet bisacodyl; fleet bisacodyl; flexeril; flexor; flextra-650; floctafenine; flo-gel; flolan; flomax; flonase; florinef; flosequinan; flovent; flovent hf a; flovent rotadisk; floxin; floxuridine; fluconazole; flucytosine; fludara; fludarabine; fludarabine phosphate; fludrocortisone; flumadine; flumazenil; flumethasone+clioquinol; flumist; flunarizine; flunisolide; fluocinolone; fluocinolone+tretinoin+hydroquinone; fluocinolone acetonide; fluogen; fluor-a-day; fluorinse; fluorometholone; fluoroplex; fluorouracil; fluoxetine; fluoxymesterone; flupentixol; fluphenazine; fluphenazine decanoate; flurandrenolide; flurbiprofen; flur-rinse; flushield; flutamide; fluticasone; fluticasone+salmeterol; fluticasone propionate; fluvastatin; fluvirin; fluvoxamine; fluvoxamine maleate; fluzone; fml; fml s.o.p.; focalin; focalin xr; folgard; folgard rx 2.2; folic acid; folic acid+nicotinamide; follistim; follitropin alfa; follitropin beta; foltrin; foltx; folvite; fomivirsen; fondaparinux sodium; foradil; formaldehyde; formalyde-10; formoterol; formoterol fumarate; formulation r; fortamet; forteo; fortovase; fosamax; fosamax plus d; fosamprenavir; fosaprepitant dimeglumine; foscarnet sodium; foscavir; fosfomycin tromethamine; fosinopril; fosinopril sodium; fosphenytoin; fosphenytoin sodium; fostex; fragmin; framycetin+gramicidin+dexamethasone; framycetin+gramicidin+phenylephrine; freamine; frova; frovatriptan; frovatriptan succinate; fudr; fulvestrant; fulvicin; fungizone; fungoid; furadantin; furazolidone; furosemide; furoxone; fusidate sodium; fusidic acid+hydrocortisone; fusidic acid+fusidate sodium; fuzeon; gabapentin; gabarone; gabitril; galantamine; galantamine bromide; gallium nitrate; galzin; gamastan; gamimune n; gammagard; gammar; ganciclovir; ganite; gantanol; garamycin; garlic; gas relief; gastrocrom; gastrografin; gas-x; gatifloxacin; gaviscon; gefitinib; gel-kam; gelusil; gemcitabine; gemcitabine hydrochloride; gemcor; gemfibrozil; gemifloxacin; gemtuzumab ozogamicin; gemzar; genahist; gengraf; genotropin; gentamicin; gentamicin w+betamethasone; gentian; geodon; geref; gerimal; germanium; ginger; ginkgo; ginseng; glatiramer; glatiramer acetate; glauctabs; gleevec; gliadel; gliclazide; glimepiride; glipizide; glipizide+metformin; glipizide xl; glucagon; glucagon recombinant; glucophage; glucophage xr; glucosamine; glucose; glucotrol; glucotrol xl; glucovance; glumetza; glyburide; glyburide+metformin; glycerin; glycolax; glycopyrrolate; glycron; glynase; glyset; gold sodium thiomalate; goldenseal; golytely; gonadotropins chorionic; gonal-f; goserelin; gramicidin+polymyxin b; granisetron; granul; granulex; grape seed; green tea; grifulvin v; grisactin; griseofulvin; gris-peg; guaifenesin; guaifenesin+guaifenesin w+pseudoephedrine; guaifenesin+antitussive+decongestant+antihist; guaifenesin+codeine+ammonium chloride; guaifenesin+decon+narc+antitussive+antihistamine; guaifenesin+decon+narc antitussive; guaifenesin+decong+narc antitussive; guaifenesin+decongestant; guaifenesin+decongestant+antihistamine; guaifenesin+dextromethorphan+decongestant; guaifenesin+narcotic antitussive+antihistamine; guaifenesin+oxtriphylline; guaifenesin+potas guaiaco+dextromethorphan; guaifenesin+potassium guaiacolsulfonate; guaifenesin+pseudoephedrine+hydrocodone; guaifenesin+theophylline+ephedrine; guaifenesin+theophylline+pseudoephedrine; guaifenesin+xanthine+decongestant+barbiturate; guaifenesin w+phenylpropanolamine; guaifenesin w+pseudoephedrine or dextromethorphan; guaifenesin+codeine; guaifenesin+dextromethorphan; guaifenesin+dextromethorphan polistirexl; guaifenesin+dyphylline; guaifenesin+ephedrine; guaifenesin+hydrocodone; guaifenesin+hydromorphone; guaifenesin+phenylephrine; guaifenesin+phenylephrine+phenylpropano; guaifenesin+pseudoephedrine; guaifenesin+theophylline; guaifenesin-carbetapentane-decongestant; guaifenex pse; guanabenz; guanadrel; guanethidine; guanethidine+hydrochlorothiazide; guanfacine; guarana; guiatuss; gyne sulf; gynodiol; habitrol; halcion; haldol; halfan; halobetasol; halofantrine; haloperidol; haloperidol decanoate; haloperidol lactate; halotestin; hamamelis leaf; harmonyl; havrix; hawthorn; hectorol; helidac; helixate; hemabate; heme iron polypeptide; heme iron polypeptide+folic acid; hemin; hemocyte-c; hemofil-m; hemorrhoidal; hemorrhoidal suppository; heparin; heparin lock flush; hep-lock; hepsera; heptalac; herceptin; hexachlorophene-cleanser; hexadrol; hexalen; hiprex; hismanal; histrelin; hivid; homatropine hydrobromide; homocysteine formula; hops; horse chestnut; humalog; humalog mix; humatin; humatrope; humegon; humibid; humibid dm; humibid la; humira; humorsol; humulin; humulin n; hurricaine; hyalgan; hyaluronate; hyaluronic acid; hyaluronidase; hyate c; hycamtin; hycodan; hycomine; hydergine; hydralazine; hydralazine+hctz; hydralazine+reserpine; hydralazine-reserpine-hctz; hydrea; hydrochlorothiazide; hydrochlorothiazide+benazepril; hydrochlorothiazide+captopril; hydrochlorothiazide+enalapril; hydrochlorothiazide+losartan; hydrochlorothiazide+triamterene; hydrochlorothiazide w+amiloride; hydrochlorothiazide w+triamterene; hydrocodone; hydrocodone+acetaminophen; hydrocodone+chlorpheniramine; hydrocodone+ibuprofen; hydrocodone+homatropine; hydrocodone+potassium guaiacolsulfonate; hydrocortisone; hydrocortisone acetate; hydrocortisone probutate; hydrocortisone sodium succinate; hydrocortisone valerate; hydrocortisone-benzoyl peroxide; hydrocortisone-iodoquinol; hydrocortisone-pramoxine-chloroxylenol; hydrocortisone-yerba santa; hydrocortone; hydrodiuril; hydrolac; hydromorphone; hydromorphone hydrochloride; hydroquinone; hydroquinone+avobenzone; hydroquinone+sunscreens; hydroquinone+paba-sunscreen; hydroxocobalamin; hydroxychloroquine; hydroxychloroquine sulphate; hydroxypropylmethylcellulose; hydroxyurea; hydroxyzine; hygroton; hylan g-f 20; hylorel; hyoscine; hyoscine butylbromide; hyoscyamine; hyoscyamine; hyoscyamine; hyoscyamine+atropine+scopolamine+phenobarbital; hyosol+sl; hyperton-5; hytakerol; hytrin; hyzaar; ibandronate; ibandronate sodium; ibandronic acid; ibritumomab tiuxetan; ibuprofen; ibuprofen+pseudoephedrine hydrochloride; ibuprofen+hydrocodone; icy hot; idamycin; idarubicin; idarubicin hydrochloride; idoxuridine; ifex; ifosfamide; iletin i nph; ilopan-choline; ilosone; ilotycin; imatinib; imatinib mesylate; imdur; imiglucerase; imipenem+cilastatin; imipramine; imiquimod; imitrex; immune globulin; imodium; imodium a-d; imodium advanced; imuran; inamrinone; inapsine; indapamide; inderal; inderal la; indinavir; indocin; indocin-sr; indomethacin; infanrix; infed; infergen; infliximab; infumorph; inh; innohep; innopran xl; inocor; inspra; insulin; insulin aspart; insulin aspart protamine+insulin aspart; insulin glargine; insulin lispro; insulin lispro protamine+insulin lispro; intal; integrilin; interferon; interferon alfa; interferon beta 1a; interferon beta 1-b; interferon gamma 1b; intralipid; intron-a; intropin; invanz; inversine; invirase; iodinated glycerol; iodoquinol; iodotope; iopidine; ipecac; ipol; ipratropium; ipratropium+albuterol; ipratropium bromide; irbesartan; irbesartan+hydrochlorothiazide; irbesartan w; irbesartan w+hydrochlorothiazide; iressa; irinotecan; irinotecan hydrochloride; iron dextran; iron sucrose; iron supplements; iron+ascorbic acid; iron+stool softener; iron+vitamin c+vitamin b12; iron-sorbitol-citric acid complex; iron-vitamin c-vitamin b12-folic acid; laxatives; ismelin; ismo; isocom; isoetharine; isoetharine mesylate; isoflurophate; isoniazid; isopropamide w+trifluoperazine; isoproterenol; isoptin; isoptin sr; isopto atropine; isopto carbachol; isopto carpine; isopto homatropine; isopto hyoscine; isordil; isosorbide dinitrate; isosorbide mononitrate; isotretinoin; isoxsuprine; isphagula+psyllium; isradipine; isuprel; itch-x; itraconazole; iveegam; ivermectin; jantoven; januvia; java tea; junel fe; kabikinase; kadian; kaletra; kanamycin sulfate; kanax xr; kantrex; kaolin+pectin; kaon cl; kaopectate; kaopectate 1-d; kaopectate advanced formula; kaopectate ii; kapectolin; karidium; karigel; kariva; kava; kayexalate; k-dur; keep alert; keflex; keftab; keppra; kerlone; ketek; ketoconazole; ketoprofen; ketorolac; ketorolac tromethamine; ketotifen; ketotifen fumarate; kie; kineret; kionex; klaron; klonopin; klonopin wafers; k-lor; klor-con; klorvess; klotrix; k-lyte cl; koate hp; kogenate; kola wine; konsyl fiber; konyne 80; koro-flex; koromex; k-pek ii; k-phos; k-phos no.2; k-phos original; k-tab; kytril; labetalol; labetalol hydrochloride; lac-hydrin; lacrisert; lactobacillus acidophilus; lactulose; lamictal; lamisil; lamivudine; lamivudine+zidovudine; lamivudine-hbv; lamotrigine; lamprene; laniazid; lanoxicaps; lanoxin; lansoprazole; lansoprazole+amoxicillin+clarithromycin; lansoprazole+naproxen; lantus; lapatinib; lariam; larodopa; lasix; latanoprost; latanoprost+timolol maleate; ledercillin vk; leflunomide; lemon balm; lenalidomide; lepirudin; lercanidipine; lescol; lescol xl; letrozole; leucovorin; leukeran; leukine; leuprolide; leustatin; levalbuterol; levalbuterol hydrochloride; levamisole; levaquin; levetiracetam; levitra; levocabastine; levocarnitine; levocetirizine; levocetirizine dihydrochloride; levodopa; levodopa+carbidopa; levofloxacin; levomethadyl; levonorgestrel; levonorgestrel+ethinyl estradiol; levophed; levora; levothroid; levothyroxine; levothyroxine sodium; levoxyl; levsin; levsin+sl; levulan; lexapro; lexiva; lexxel; librax; librium; licorice; lida mantle hc; lidamantle; lidocaine; lidocaine+hydrocortisone; lidocaine+prilocaine disc; lidocaine w+prilocaine; lidoderm; lidopain; limbitrol; lincocin; lincomycin; lindane; linezolid; lioresal; liothyronine sodium; liotrix; lipisorb; lipitor; liposyn; liquaemin; liqui-citra; liquid pred; liqui-flur; liquimat; liquiprin; lisinopril; lisinopril+hydrochlorothiazide; lisinopril w; lisinopril w+hydrochlorothiazide; lithium; lithium carbonate; lithobid; lithonate; lithostat; lithotabs; livostin; lobac; lodine; lodine xl; lodosyn; lodoxamide; loestrin 24 fe; loestrin fe; lomefloxacin; lomotil; lomustine; loniten; loperamide; loperamide+simethicone; lopid; lopinavir+ritonavir; lopressor; lopressor hct; loprox; lorabid; loracarbef; loratadine; loratadine+pseudoephedrine; lorazepam; lorcet; lorcet plus; lortab; losartan; losartan+hydrochlorothiazide; losartan potassium; losartan potassium+hydrochlorothiazide; losartan w; losartan w+hydrochlorothiazide; lotemax; lotensin; lotensin hct; loteprednol; loteprednol etabonate; lotrel; lotrimin; lotrisone; lotronex; lovastatin; lovaza; lovenox; low-ogestrel; loxapine; loxitane; lozol; lubiprostone; ludiomil; lufyllin; lufyllin-gg; lumigan; lunelle; lunesta; lupron; luride; lustra; luvox; luxiq; lymerix; lyphocin; lypressin; lyrica; lysodren; lysporin; maalox; maalox anti-diarrheal; maalox antidiarrheal caplets; macrobid; macrodantin; mafenide acetate; magaldrate; magic bullet; magnacaps; magnesium citrate; magnesium hydroxide; magnesium hydroxide-cascara sagrada; magnesium salicylate; magnesium salicylate-phenyltoloxamine; magnesium sulfate; magnesium supplement; mag-ox 400; magsal; ma-huang; malarone; malathion; mandelamine; mannitol; mao inhibitors; maprotiline; marax; marcaine; marezine; marigold; marinol; marlexate; masoprocol; matulane; mavik; max epa; maxair; maxair autohaler; maxalt; maxalt mlt; maxaquin; maxifed; maxipime; maxitrol; maxitrol; maxolon; maxzide; maxzide; m-clear; md-gastroview; mebendazole; mecamylamine; mechlorethamine; meclan; meclizine; meclizine+nicotinic acid; nicotinic acid+lavopiprant; meclizine+cyclizine; meclocycline; meclofenamate; meclomen; medi-lice; medipren; medrogestone; medrol; medroxyprogesterone; medroxyprogesterone acetate; med-rx; med-rx dm; mefenamic acid; mefloquine; mega b-12; megace; megestrol; megestrol acetate; melanex; melatonin; mellaril; meloxicam; melphalan; melphalan hydrochloride; memantine; menest; meni-d; menogen; menostar; menotropins; mentax; mepenzolate; mepergan fortis; meperidine; mephenytoin; mephyton; meprobamate; mepron; meprozine; mercaptopurine; meridia; meropenem; merrem; meruvax ii; mesalamine; mesantoin; mesna; mesnex; mesoridazine; mestinon; metadate cd; metaglip; metaproterenol; metastron; metaxalone; metformin; metformin hydrochloride; metformin hydrochloride+pioglitazone hydrochloride; methacholine; methadone; methadose; methamphetamine hydrochloride; methazolamide; methenamine; methenamine+methylene blue+benzoic+phenyl sal+at; methenamine mandelate-sodium acid phosphate; methenamine-meth blue-salicylate-sod biphos; methergine; methimazole; methocarbamol; methocarbamol+aspirin+codeine; methocarbamol+ibuprofen; methocarbamol+aspirin; methocarbamol-acetaminophen; methohexital; methotrexate; methotrimeprazine; methoxsalen; methscopolamine; methsuximide; methyl salicylate+menthol; methyldopa; methyldopa w+thiazide diuretics; methylene blue; methylergonovine; methylin; methylphenidate; methylphenidate hydrochloride; methylprednisolone; methylprednisolone acetate; methylprednisolone sodium succinate; methylprednisolone-neomycin compound; methylsulfonylmethane; methyltestosterone; methyltestosterone+esterified estrogens; methyltestosterone+estrogen; methysergide; metipranolol; metoclopramide; metolazone; metopirone; metoprolol; metoprolol succinate; metoprolol tartrate; metoprolol+hctz; metro; metrocream; metrodin; metrogel; metrolotion; metronidazole; metyrapone; mevacor; mexiletine; mexitil; miacalcin; mibef radii; micafungin sodium; micardis; micardis hct; micatin; micomp-pb; miconazole; micrainin; micronase; micronor; microzide; midamor; midazolam; midchlor; midodrine; midodrine hydrochloride; midrin; mifeprex; mifepristone; migergot; miglitol; migranal; milk of magnesia w+cascara; milk thistle; milrinone; milrinone lactate; miltown; minipress; minitran; minizide; minocin; minocycline; minocycline hydrochloride; minoxidil; mintezol; miradon; miralax; mirapex; mirena; mirtazapine; misoprostol; misoprostol+diclofenac; mithracin; mitomycin; mitotane; mitoxantrone; mitoxantrone hydrochloride; mitrolan; m-m-r ii; moban; mobic; mobigesic; moclobemide; modafinil; moduretic; moexipril; moexipril hydrochloride; moexipril-hydrochlorothiazide; molindone; momentum; mometasone; mometasone furoate; monistat; monobenzone; monoket; monopril; montelukast; montelukast granules; monurol; moricizine; morphine; morphine sulfate; motofen; motrin; moxifloxacin; moxifloxacin hydrochloride; m-oxy; mucinex; mucomyst; multivitamin-mineral-antioxidant; multivitamins; multivitamins+iron+fluoride; multivitamins+fluoride; multivitamins+iron; mupirocin; muse; mustargen; mutamycin; myambutol; mycelex; myciguent; mycitracin; mycobutin; mycolog ii; mycophenolate mofetil; mycophenolate mofetil hydrochloride; mycophenolate sodium; mycostatin; myco-triacet ii; mydral; mydriacyl; mykrox; mylanta; myleran; mylicon; mylotarg; myochrysine; mysoline; mytelase; nabilone; nabumetone; nadolol; nafarelin; nafarelin acetate; nafrinse; naftifine; naftin; nalbuphine; naldecon; nalfon; nalidixic acid; nalmefene; naloxone; naltrexone hcl; namenda; naphazoline; naphazoline+antazoline; naphazoline+pheniramine; naphcon; naprelan; naprosyn; naproxen; naratriptan; naratriptan hydrochloride; narcan; narcotic analgesic+apap+caffeine; narcotic analgesic+aspirin+caffeine; narcotic analgesic-apap-caffeine-butalbital; narcotic analgesics; narcotic analgesics+acetaminophen; narcotic analgesics+aspirin; narcotic antitussive+antihistamine; naropin; nasacort; nasalcrom; nasalide; nasarel; nascobal; nasonex; natalizumab; nateglinide; natrecor; navane; navelbine; nebcin; nebivolol hydrochloride; nebupent; necon; nedocromil sodium; nefazodone; neggram; nelfinavir; nembutal; neo-fradin; neomycin; neomycin+bacitracin+polymyxin; neomycin+colistin+hydrocortisone; neomycin+polymyxin+steroid; neomycin+bacitracin+polymyxin+hydrocortisone; neomycin+colistin+hydrocortisone+thonzonium; neomycin+gramicidin+nystatin+triamcinolone; neomycin+polymyxin b+gramicidin d; neomycin+polymyxin+dexamethasone; neomycin+polymyxin+hydrocortisone; neopap; neoral; neosar; neosporin; neostigmine bromide; neo-synephrine; neo-tabs; neotricin hc; neptazane; nesiritide; nestrex; netilmicin sulfate; netromycin; nettle; neulasta; neumega; neupogen; neuro-k; neurontin; neutracare; neutra-phos; neutrexin; nevirapine; nexium; niacin; niacin+lovastatin; niacin+simvastatin; niacor; niaspan; niazid; nicardipine; nicobid; nicoderm; nicolar; nicomide; nicorette; nicosyn; nicotine; nicotinic acid; nicotrol; nifedipine; niferex; nilandron; nilotinib; nilstat; nilutamide; nimodipine; nimotop; nipent; niravam; nisoldipine; nitazoxanide; nitisinone; nitrates; nitrazepam; nitro-bid; nitrodisc; nitro-dur; nitrofurantoin; nitrofurantoin+nitrofurantoin macrocrystals; nitrogard; nitroglycerin; nitroglycerin la; nitrol; nitrolingual; nitropress; nitrostat; nix; nizatidine; nizoral; no doz; nolvadex; nonsteroidal antiinflammatory drugs; norco; norcuron; norditropin; norepinephrine bitartrate; norethindrone; norethindrone+ethinyl estradiol; norethindrone acetate+ethinyl estradiol w+ferrous; norflex; norfloxacin; norgesic; norgestrel; noritate; normiflo; normocarb; normocarb 25 bicarbonate; normodyne; noroxin; norpace; norplant; norpramin; nor-q-d; nortriptyline; norvasc; norvir; novacet; novafed; novagesic; novantrone; novolin; novolog; novoseven; nubain; nulev; nulytely; numonyl; nuprin; nutrilipid; nutropin; nuvaring; nybcen; nydrazid; nylidrin; nystatin+metronidazole; nystatin+triamcinolone; nystex; obenix; oby-trim; ocean; octamide; octreotide; octreotide acetate; ocufen; ocuflox; ocupress; ocusert pilo; ocutricin-hc; ofloxacin; ogen; olanzapine; olanzapine+fluoxetine; olmesartan medoxomil; olmesartan medoxomil+hydrochlorothiazide; olopatadine; olopatadine hydrochloride; olsalazine; olux; omalizumab; omega-3; omeprazole; omeprazole+sodium bicarbonate; omeprazole magnesium; omnicef; omnipen; oncaspar; oncovin; ondansetron; ontak; opcon-a; ophthetic; opium+kaolin+pectin+belladonna; oprelvekin; opti-clear; opticrom; opticyl; optipranolol; optivar; oracit; oral contraceptives; oralone; orap; orapred; orasone; orazinc; ordrine at; orencia; orfadin; organidin nr; orgaran; orimune; orinase; orlaam; orlistat; orphenadrine; orphenadrine+aspirin+caffeine; orphenate; ortho evra; ortho tri-cyclen; ortho-diaphragm; ortho-dienestrol; orudis; oruvail; os-cal; oseltamivir; osmitrol; otimar; otocain; otocort; otogesic; otomar-hc; ovace; ovcon 35; ovide; ovidrel; ovrette; oxaliplatin; oxaprozin; oxazepam; oxcarbazepine; oxiconazole nitrate; oxistat; oxprenolol; oxsoralen; oxsoralen-ultra; oxybutynin; oxybutynin chloride; oxybutynin er; oxycodone; oxycodone+acetaminophen; oxycodone hydrochloride; oxycodone hydrochloride+acetaminophen; oxycontin; oxydose; oxyfast; oxyir; oxymetholone; oxytocin; oxytrol; p1e1; paba; paclitaxel; pain relievers; paliperidone; palivizumab; palladone; palmitate-a; palonosetron; palonosetron hydrochloride; pamelor; pamidronate; pamidronate disodium; pamine; pamine forte; panadol; panafil; pancrease; pancrelipase; pancron; pandel; panhematin; panixine; panretin; pantoprazole; pantoprazole+edta; pantoprazole magnesium; pantoprazole sodium; papain+urea; papaverine; papaya; paradione; paraflex; parafon forte; paramethadione; paraplatin; parcopa; parepectolin; paricalcitol; parlodel; paromomycin; paroxetine; paroxetine hydrochloride; paroxetine mesylate; passion flower; patanol; pavabid; paxil; paxil cr; pc-cap 65; pce; pediacof; pediacon ex; pediaflor; pedialyte; pediapred; pediazole; pediazole; pediotic; pedituss; pegademase; peganone; pegaspargase; pegasys; pegfilgrastim; peginterferon alfa 2a; peg-intron; pegvisomant; pemadd; pemetrexed sodium; pemirolast; pemoline; penciclovir; penetrex; penicillamine; penicillin g benzathine; penicillin v; penicillin v potassium; penicillins; penlac; pentacarinat; pentam 300; pentamidine isethionate; pentasa; pentazocine; pentazocine+acetaminophen; pentazocine w+naloxone; pentobarbital; pentolair; pentosan; pentosan polysulfate sodium; pentostatin; pentoxifylline; pentrax; pentuss; pen-vee k; pepcid; peppermint oil; pepto diarrhea control; pepto-bismol; percocet; percolone; perdiem; perflutren; pergolide; pergonal; pericyazine; peridex; perindopril; perindopril erbumine; perindopril erbumine+indapamide; periogard; periostat; permax; permethrin; perphenazine; persa-gel; persantine; persantine iv; pexeva; phazyme; phenacemide; phenazopyridine; phenazopyridine-hyoscyamine-butabarbital; phenergan; phenergan codeine; phenergan vc w+codeine; phenergan w+codeine; phenergan w+dextromethorphan; phenobarbital; phenothiazines; phenoxybenzamine; phensuximide; phentermine; phentolamine mesylate; phen-tuss; phen-tuss ad; phen-tuss dm; phenurone; phenylbutazone; phenylbutazone+oxyphenbutazone; phenylephrine; phenylephrine+codeine+chlorpheniramine+potassium; phenylephrine+shark liver oil+mineral oil+petrol; phenylpropanolamine; phenylpropanolamine+hydrocodone; phenytoin; phenytoin sodium; phenytoin w+phenobarbital; phisohex; phos-flur; phoslo; phospholine iodide; photofrin; phrenilin; physostigmine; pilocar; pilocarpine; pilocarpine+epinephrine; pilopine hs; piloptic-1; pima; pimecrolimus; pimozide; pinaverium; pinaverium bromide; pink bismuth; pin-rid; pin-x; pioglitazone; pioglitazone hydrochloride; piperacillin+tazobactam; piperacillin+tazobactam sodium; pirbuterol; pirenzepine; piroxicam; pitocin; pivampicillin; pizotifen; placidyl; plan b; plaquenil; plasbumin; platinol-aq; plavix; plendil; pletal; plicamycin; pocion jaccoud; podofilox; pollen; poly otic; polycarb; polycarbophil; polycillin; polycitra-k; poly-dex; polyethylene glycol; polygam; polymox; polymyxin b+trimethoprim; polymyxin b sulfate; polysaccharide iron complex; polytinic; polytrim; ponstel; pontocaine; porfimer sodium; posaconazole; posicor; potaba; potas guaico+phenylephrine+codeine+prometh; potassium acid phosphate; potassium chloride; potassium citrate; potassium guaiacolsulfonate-decongestant; potassium guaico+codeine+promethazine; potassium iodide; potassium iodide w+theophylline; potassium iodide-ephedrine expectorant; potassium iodide-xanthine-decongestant-barbiturate; potassium para-aminobenzoate; potassium permanganate granules; potassium supplements; ppis; pramipexole; pramipexole dihydrochloride; pramlintide; pramlintide acetate; pramosone; pramoxine+benzyl alcohol; pramoxine+hydrocortisone; pramoxine+camphor+calamine; pramoxine+camphor+zinc acetate; prandin; pravachol; pravastatin; pravastatin+buffered aspirin; pravastatin sodium; pravigard pac; praziquantel; prazosin; prazosin+polythiazide; precose; prednicen-m; prednisolone; prednisolone acetate; prednisolone sodium phosphate; prednisone; prednisone intensol; pregabalin; pregabalin+lyrica; pregnyl; prelone; premarin; premarin tabs; premarin w+methyltestosterone; premesis rx; premphase; prempro; prenatal vitamins+iron-folic acid; prenatal vitamins-minerals-omega-3 fatty acids; prentif cavity-rim cery cap; preparation h; prevacid; preven; prevident; prevnar; prevpac; priftin; prilosec; primacare one; primacor; primaquine; primaquine phosphate; primaxin; primidone; principen; prinivil; prinizide; prinzide; proair hfa; proamatine; pro-banthine; probenecid; probucol; procainamide; procalamine; procanbid; procan-sr; procarbazine; procardia; procardia xl; prochlorperazine; procrit; proctocort; proctocream-hc; proctofoam-hc; proctosol hc; profasi; proferrin; progestasert; progesterone; progestone; proglycem; prograf; proguanil; prolastin; proleukin; prolex dh; prolex dm; prolixin; proloprim; promethazine; promethazine+codeine; promethazine w+dextromethorphan; promethazine+meperidine; promethazine+phenylephrine; promethazine+phenylephrine+codeine; promethegan; prometrium; pronestyl; pronestyl-s.r.; pronto; propafenone; propafenone hydrochloride; propantheline; proparacaine; propecia; propionate+amino acid+urea; proplex t; propoxyphene; propoxyphene+aspirin+caffeine; propoxyphene+acetaminophen; propoxyphene hcl+acetaminophen; propoxyphene napsylate+acetaminophen; propoxyphene w+aspirin; propranolol; propranolol s.r.; propulsid; propylene glycol+polyethylene glycol; propylthiouracil; proquin xr; proscar; prosed+ds; prostigmin; prostin e2; prostin yr pediatric; protilase; protonix; protonix iv; proton-pump inhibitors; protopic; protropin; proventil; proventil hfa; provera; provigil; provocholine; prozac; pseudoephedrine; pseudoephedrine+cetirizine; pseudoephedrine+fexofenadine; pseudoephedrine+loratidine; pseudoephedrine w+terfenadine; pseudovent 400; psoriatec; ptu; pulmicort; pulmicort respules; pulmicort turbuhaler; pulmozyme; purinethol; pygeum; pyrantel; pyrazinamide; pyrethrins+piperonyl butoxide; pyridium; pyridostigmine; pyridoxine; pyrimethamine; pyrin-x; pyrinyl ii; qm-260; quadramet; questran; quetiapine; quetiapine fumarate; quibron; quinaglute; quinapril; quinapril+hydrochlorothiazide; quinapril hcl+hydrochlorothiazide; quindal; quinerva; quinidex; quinidine; quinine; quinine sulfate; quinite; quinora; quinupristin-dalfopristin; quixin; qvar; rabeprazole; rabeprazole sodium; raloxifene; raloxifene hydrochloride; ramelteon; ramipril; ramipril+hydrochlorothiazide; raniclor; ranitidine; ranitidine bismuth citrate; ranitidine hydrochloride; ranolazine; rapamune; rapinex; raptiva; rasagiline; rasagiline mesylate; rasburicase; rauwolfia alkaloids+thiazide diuretics; razadyne; razadyne er; rebetol; rebetron; rebif; reclast; recombinate; recombivax hb; rectacaine; rectocort-hc; red clover; red yeast rice; redux; refludan; reglan; regranex; relafen; relaxazone; relenza; relpax; remeron; remeron soltab; remicade; remifentanil; reminyl; remodulin; remular-s; renagel; renova; reopro; repaglinide; repaglinide+metformin; requip; rescaps-d; rescriptor; rescula; reserpine; respbid; respigam; respiratory syncytial virus immune globulin; restasis; restoril; restylane; retavase; reteplase; retin-a; retrovir; revatio; revex; rev-eyes; revia; reyataz; rheaban; rheumatrex; rhinaris; rhinocort; rhinocort aqua; rhinoflex; ribavirin; ricotuss; ridaura; rifabutin; rifadin; rifamate; rifampin; rifampin+isoniazid-pyrazinamide; rifampin+isoniazid; rifapentine; rifater; rifaximin; rilutek; riluzole; riluzole; rimactane; rimantadine; rimexolone; riomet; riopan; risedronate; risedronate sodium; risedronate sodium+calcium; risperdal; risperdal consta; risperdal m; risperidone; ritalin; ritalin la; ritalin sr; ritonavir; rituxan; rituximab; rivastigmine; rizatriptan; rizatriptan benzoate; robaxin; robaxisal; robinul; robitussin; robitussin a-c; robitussin-dm; rocaltrol; rocuronium bromide; rodex td; rofecoxib; roferon-a; rogaine; rolaids; romazicon; ronoxidil; ropinirole; ropinirole hydrochloride; ropivacaine; rosanil cleanser; rosiglitazone; rosiglitazone+metformin; rosiglitazone maleate+glimepiride; rosiglitazone maleate+metformin; rosula; rosuvastatin; rotigotine; rowasa; roxicet; roxicodone; roxicodone intensol; rozerem; rozex; rubex; rythmol; rythmol sr; sage leaf; salagen; salbutamol; salbutamol sulfate+ipratropium; salflex; salicylamide+acetaminophen+phenyltoloxamine capsul; salicylic acid or salicylic acid combinations; salicylic-sulfur; salmeterol; salmeterol+fluticasone; salmon oil; salonpas-hot; salsalate; sal-tropine; samarium sm-153+lexidronam; sanctura; sandimmune; sandoglobulin; sandostatin; sandostatin lar; sani-supp; sansert; santyl; saquinavir; saquinavir mesylate; sarafem; sargramostim; saw palmetto; schisandra; scopolamine; sea mist; sea-clens; seasonale; seba-nil; sebex; sebulex; sectral; seldane; seldane-d; selegiline; selenium sulfide; selsun blue; senna; senna+psyllium seed; sennosides a+b; senokot; sensorcaine; septisol; septra; ser-ap-es; serax; serentil; serevent; sermorelin; seromycin; serophene; seroquel; serostim; serpazide; sertraline; sertraline hydrocloride; serzone; sevelamer; sevelamer carbonate; sevoflurane; shark cartilage; shohl's; sibutramine; sibutramine hydrochloride monohydrate; sildenafil; sildenafil citrate; silvadene; silver sulfadiazine; simethicone; simulect; simvastatin; simvastatin+ezetimibe; sinemet; sinemet cr; sinequan; singulair; sinupan; sirolimus; sitagliptin; skelaxin; skelid; slo-bid; slo-niacin; slo-phyllin; slo-phyllin gg; slow-k; sochlor; sodium bicarbonate; sodium chloride; sodium citrate; sodium clodronate; sodium ferric gluconate; sodium ferric gluconate complex; sodium fluoride; sodium fluoride+karidium+karigel+neutracare; sodium iodide i 131; sodium nitroprusside; sodium phenylbutyrate; sodium phosphate monobasic-dibasic; sodium phosphate-potassium phosphate; sodium polystyrene sulfonate; sodium sulfacetamide; sodium thiosulfate; sodium-potassium citrates; solage; solaraze; solganal; solifenacin succinate; solu-cortef; solu-medrol; solurex; soma; soma compound; soma compound w+codeine; somatrem; somatropin; somavert; sominex; sonata; sorbitol; sorbitrate; soriatane; sotalol; sotret; soy; sparfloxacin; spectinomycin; spectracef; spectro-max; spiramycin; spiriva; spironolactone; sporanox; sprintec; sps; sski; st. john's wort; stadol; stadol ns; staflex; stalevo; stanimax; stannous fluoride; starlix; staticin; statins; stavudine; stay awake; steroid+antibiotic; stimate; stool softeners+stimulant combination laxatives; strattera; streptase; streptokinase; streptozocin; striant; stromectol; strontium-89 chloride; sublimaze; suboxone; subutex; succimer; sucralfate; sudafed; sudal; sufenta; sufentanil; sular; sulfacetamide+prednisolone; sulfacetamide sodium; sulfacetamide+sulfur; sulfacetamide-urea; sulfacet-r; sulfadiazine; sulfadiazine+trimethoprim; sulfadoxine+pyrimethamine; sulfamethoxazole; sulfamethoxazole+trimethoprim; sulfamylon; sulfanilamide; sulfasalazine; sulfas-phenylpropanolamine-pyrilamine-pheniramine; sulfathiazole-sulfacetamide-sulfabenzamide; sulfinpyrazone; sulfisoxazole+phenazopyridine; sulfonamides; sulfur-alcohol; sulfuric acid+phenolics; sulindac; sultrin; sultrin triple sulfa; sumatriptan; sumatriptan succinate; sumatriptan+naproxen sodium; sumycin; sunitinib malate; sunkist; sunscreens; superepa; supprelin; suprax; surbex; surfak; surmontil; sustiva; sweet clover; symadine; symbyax; symlin; symmetrel; synagis; synarel; synercid; syn-rx; syn-rx dm; synthroid; synvisc; tacrine; tacrolimus; tadalafil; tagamet; tagamet hb; talacen; talwin nx; tambocor; tamiflu; tamoxifen; tamoxifen citrate; tamsulosin; tamsulosin hydrochloride; tannate 12 s; tannic-12; tannihist-12 rf; tao; tapazole; tarabine pfs; tarceva; targretin; tarka; tasmar; tavist; taxol; taxotere; tazarotene; tazorac; tea tree oil; teczem; tedrigen; tegaserod; tegison; tegretol; tegretol xr; tegrin; tegrin lf; telithromycin; telmisartan; telmisartan+hydrochlorothiazide; temazepam; temodar; temovate; temozolamide; temozolomide; tempo; temsirolimus; tenex; teniposide; ten-k; tenofovir; tenoretic; tenormin; tequin; terazol; terazosin; terbinaffne; terbinafine hydrochloride; terbutaline; terconazole; terconazole+miconazole; terfenadine; teriparatide; terpin hydrate+codeine; tesamone; tessalon perle; tessalon perles; testim; testoderm; testoderm tts; testosterone; testosterone undecanoate; testred; testro; tetanus toxoid; tetracaine; tetracycline; tetracycline+metronidazole+bismuth subsalicylate; tetracyclines; tetrahydrozoline; tetramune; thalidomide; thalomid; thalomide; theo-24; theocon; theo-dur; theolair; theomar gg; theophen; theophylline; theophylline+ephedrine+hydroxyzine; theophylline+ephedrine+phenobarbital; theophylline+ephedrine+phenobarbital long acting; theophylline+iodinated glycerol; theotal; theracys; thera-flur; theravite; thiabendazole; thiamilate; thiamine; thiazide diuretics; thiazide w+spironolactone; thiethylperazine; thioguanine; thiola; thioplex; thioproperazine; thioridazine; thiotepa; thiothixene; thorazine; thyme; thymoglobulin; thyroid replacement; thyroid+armour; thyrolar; thyrotropin; tiagabine; tiagabine hydrochloride; tiaprofenic acid; tiazac; tibolone; ticarcillin+clavulanate potassium; tice bcg; ticlid; ticlopidine; tigan; tigecycline; tikosyn; tilade; tiludronate; timentin; timolol; timolol maleate; timolol-dorzolamide; timoptic; timoptic-xe; tin-ben; tincture of opium; tinzaparin; tioconazole; tiopronin; tiotropium; tiotropium bromide monohydrate; tipranavir; tirofiban; tisit; titralac; tizanidine; tobi; tobradex; tobramcyin; tobramycin+dexamethasone; tobrex; tocainide; tofranil; tolazamide; tolbutamide; tolcapone; tolectin; tolfrinic; tolinase; tolmetin; tolterodine; tolterodine tartrate; tonocard; topamax; topamax sprinkle; topic form hc; topicort; topicort lp; topiramate; toposar; topotecan; topotecan hydrochloride; toprol xl; toradol; torecan; toremifene; tornalate; torsemide; tpa; tracleer; tragacanth; tramadol; tramadol+acetaminophen; tramadol hydrochloride; tramadol hydrochloride+acetaminophen; trandate; trandolapril; trandolapril hydrochloride; trandolapril hydrochloride+verapamil; trandolapril-verapamil er; tranexamic acid; transderm-nitro; transderm-scop; tranxene sd; tranxene t; trastuzumab; travasol; travatan; travoprost; trazodone; trecator sc; trelstar depot; trelstar la; trental; treprostinil; tretinoin; tretinoin+mequinol; trexall; triacetin; triamcinolone; triamcinolone acetonide; triaminic; triamterene; triamterene+hydrochlorothiazide; triavil; triavir; triaz; triazolam; triban; tricor; tricyclic antidepressants; tridil; tridione; trifluridine; trilafon; trileptal; trilisate; tri-luma; trilyte; trimebutine; trimebutine ampul; trimethadione; trimethobenzamide; trimethoprim; trimethoprim+sulfamethoxazole; trimetrexate glucuronate; trimipramine; trimox; trimpex; trinessa; trinsicon; trionate; trioxsalen; tripedia; triple antibiotic; triple sulfas-phenylpropanolamine-pheniramine; triptorelin; trisenox; trisoralen; tri-sprintec; tritec; trivora-28; trizivir; trobicin; trolamine polypeptide oleate; troleandomycin; tropicamide; trospium; trospium chloride; trovafloxacin; trovafloxacin mesylate; trovan; truphylline; trusopt; truvada; trypsin-balsam peru-castor oil; tryptophan; trysul; t-stat; tums; tuss vernade; tussi-12; tussi-12 s; tussi-12d; tussi-12d s; tussigon; tussin pediatric; tussionex; tussizone-12 rf; tusso-gest; twinrix; tylenol; tylenol+codeine; tylox; tympagesic; tysabri; ultane; ultiva; ultracet; ultram; ultram er; ultrase; ultravate; unasyn; uni-dur; uni-hist; uniphyl; uniretic; uni-serp; unithroid; univasc; unoprostone isopropyl; uracil mustard; urecholine; urelle; uretron; urex; uridon; urimar-t; urised; urispas; urkaf-pb; urocit-k; urofollitropin; urogesic; urogesic-blue; urokinase; urolene blue; uro-mag; uroqid acid no. 2; uroxatral; urso; ursodiol; vagifem; vagistat-1; valacyclovir; valacyclovir hydrochloride; valcyte; valdecoxib; valerian; valganciclovir; valganciclovir hydrochloride; valium; valproate sodium; valproic acid; valrubicin; valsartan; valsartan+hydrochlorothiazide; valsartan w+hydrochlorothiazide; valstar; valtrex; vanachol; vancenase; vanceril; vancocin; vancomycin; vancomycin+d5w; vancomycin hydrochloride; vaniqa; vanoxide; vanoxide-hc; vantin; vaqta; vardenafil; vardenafil hydrochloride; varenicline tartrate; varenicline+chantix; vasal; vascor; vaseretic; vasocidin; vasoclear; vasocon; vasocon-a; vasodilan; vasolate; vasotec; vasotec iv; v-cillin k; vecuronium bromide; veetids; velban; velcade; velosef; velsar; venbid; venlafaxine; ventolin; vepesid; verapamil; verapamil hs; verapamil hydrochloride; verelan; verelan pm; vermox; versed; verteporfin; vesanoid; vesicare; vexol; vfend; viadur; viagra; vibal; vibramycin; vicks 44; vicks dayquil; vicodin; vicodin es; vicon-c; vicoprofen; vidarabine; videx; videx ec; vigabatrin; vigamox; vinblastine; vincasar; vincristine; vinorelbine; vinorelbine tartrate; viocort; vioxx; vira-a; viracept; viramune; virazole; viread; virilon; viritab; viroptic; visicol; visine; vistamine; vistaril; vistide; visudyne; vitamin a; vitamin b complex; vitamin b-12; vitamin b12+folic acid+vitamin b6; vitamin b3; vitamin b-6; vitamin c; vitamin d; vitamin e; vitamin k; vitrasert; vitravene; vitron-c; vivarin; vivelle; vivelle-dot; vivotif berna; voltaren; voltaren-xr; voriconazole; vortex; vosol; vosol hc; vumon; vytone; vytorin; wakespan; warfarin; welchol; wellbutrin; wellbutrin sr; wellbutrin xl; westcort; wigraine; wild yam; willow bark; wingel; witch hazel; wormwood; wycillin; wydase; wygesic; wymox; wytensin; xalatan; xanax; xanax xr; xanthine derivatives; xanthinol niacinate; xeloda; xenical; xerac ac; xigris; xolair; xopenex; xopenex hfa; xylocaine; xylocaine viscous; yasmin 28; yaz; yerba mate; yocon; yodoxin; yohimbe; yohimbine; yohimex; zaclir; zaditor; zafirlukast; zagam; zalcitabine+ddc; zaleplon; zamicet; zanaflex; zanamivir; zanosar; zantac; zarontin; zaroxolyn; zavesca; zazole; z-cof 12dm; z-cof 8 dm; z-cof dm; z-cof dmx; z-cof hc; z-cof hcx; z-cof la; z-cof lax; zeasorb af; zebeta; zebutal; zegerid; zelapar; zelnorm; zemplar; zemuron; zenapax; zenchent; zephrex; zephrex la; zerit; zerit xr; zerlor; zestoretic; zestril; zetar; zetia; zevalin; ziac; ziagen; ziana; ziconotide; zidovudine; zidovudine+zdv; zilactin; zilactin-b; zilactin-l; zileuton; zinacef; zinc; zinc chloride; zinc gluconate; zinc oxide; zinc salts; zinc sulfate; zincate; zinecard; zingo; zinotic; zinx; zinx allergy; zinx cold; zinx congestion; zinx cough; ziprasidone; ziprasidone hydrochloride; zircon-f; ziroxide; zithromax; zmax; znlin; znp; zocor; zoderm; zoderm redi-pads; zofran; zofran odt; zoladex; zoledronic acid; zolene hc; zolinza; zolmitriptan; zoloft; zolpidem; zometa; zomig; zomig-zmt; zonalon; zone-a; zone-a forte; zonegran; zoneperfect omega 3; zonisamide; zorbtive; zorcaine+epinephrine; zorprin; zostavax; zostrix; zostrix hp; zostrix neuropathy; zostrix-hp; zosyn; zotane hc; zotex; zotex dmx; zotex-gp; zotex gpx; zotex lax; zotex pediatric; zotex-12d; zotex-la; zoto hc; zovia; zovirax; ztuss; ztuss dm; ztuss expectorant; ztuss zt; zyban; zydone; zyflo; zyflo cr; zylet; zyloprim; zymar; zyprexa; zyprexa zydis; zyrphen; zyrtec; zyrtec childrens; zyrtec childrens hives; zyrtec hives relief; zyrtec syrup; zyrtec-d; zytopic; zyvox.

A quantitative analysis of doctors' atorvastatin quantitative prescribing habits revealed that the most commonly prescribed quantity of 10 mg of atorvastatin is 30 unit doses or a multiple thereof. The analysis also revealed that the most commonly prescribed quantity of 20 mg of atorvastatin is 30 unit doses or a multiple thereof. The analysis also revealed that the most commonly prescribed quantity of 40 mg of atorvastatin is 30 unit doses or a multiple thereof. The analysis also revealed that the most commonly prescribed quantity of 80 mg of atorvastatin is 30 unit doses or a multiple thereof. In each case more than 50% of all prescriptions written were for 30 unit doses or a multiple thereof.

The analysis also revealed that the most common number of unit doses of atorvastatin having a strength of 10 mg prescribed in an individual prescription is 30 unit doses or a multiple thereof.

The analysis also revealed that the most common number of unit doses of atorvastatin having a strength of 20 mg prescribed in an individual prescription is 30 unit doses or a multiple thereof.

The analysis also revealed that the most common number of unit doses of atorvastatin having a strength of 40 mg prescribed in an individual prescription is 30 unit doses or a multiple thereof.

The analysis also revealed that the most common number of unit doses of atorvastatin having a strength of 80 mg prescribed in an individual prescription is 30 unit doses or a multiple thereof.

The present invention provides a kit comprising precisely 30 unit dose compartments, each unit dose compartment comprising precisely one unit dose of atorvastatin having a strength selected from the group consisting of 10 mg, 20 mg and 40 mg wherein each unit dose in each of the 30 unit dose compartments has the same strength. Such a kit may combine the advantages of one particular variety of packaging with the advantages of ease of dispensing. For example, 30 unit doses of atorvastatin may be provided in a blister sheet thereby providing the benefits of quicker atorvastatin counting, reduced atorvastatin handling, reduced materials required by the pharmacist (e.g. no additional vial is required), increased shelf life, increased patient compliance, reduced spillage and/or other advantages while at the same time reducing the requirement for having to break up the blister sheet. Such a kit will be effective at delivering such advantages in more than 50% of all atorvastatin prescriptions for 10 mg, 20 mg and 40 mg.

In the event that a prescription is written that comprises a number of unit doses of atorvastatin that is not evenly divisible by 30, it may be beneficial to interspace the precisely 30 unit dose compartments by perforations. Such perforations may be provided such that tearing along all of the perforations would result in the physical separation of each of the unit doses from each other. In other words, after tearing the perforations all of the unit doses would be provided in unit dose compartments that are not physically connected. Such perforations may also provide benefits to the consumer of the atorvastatin so that a single dose may be separated from the remaining doses while having all of the doses, separated or not, remain packaged.

In addition to the perforations, it may be beneficial to provide the precisely 30 unit dose compartments in three sheets, each one of the three sheets having 10 unit dose compartments. The 10 unit dose compartments may also be interspaced by perforations in the same manner as described above.

The unit dose compartments in a single kit should each contain atorvastatin of the same strength. For example, if one unit dose compartment in the kit contains a unit dose of atorvastatin of 10 mg, then all of the compartments in the kit will contain a unit dose of atorvastatin of 10 mg or be empty. For clarity, according to an illustrative embodiment of the invention, a kit for 20 mg of atorvastatin may comprise 30 unit doses of 20 mg atorvastatin and zero unit doses of any other strength. A particular, non-limiting example is a blister sheet having 10 unit dose cavities each containing a unit dose of 40 mg of atorvastatin and three of such blister sheets (i.e. each sheet having unit doses of 40 mg in each unit dose cavity) will be in the kit.

The unit dose compartments do not have to be the same size and/or shape. A kit according to the present invention may comprise precisely 30 unit dose compartments wherein the size of each of the 30 unit dose compartments may be different or the same. Typically, the size and shape of the unit dose compartments are the same and are dependent on the size of the unit dose that is contained within the unit dose compartment. Methods for preparing unit dose compartments having a suitable size and shape for a particular unit dose or unit dosage form are known in the art.

Methods of packaging atorvastatin comprising separating 30 unit doses of atorvastatin into 30 unit dose compartments, wherein each one of the 30 unit dose compartments each holds only one of the 30 unit doses of atorvastatin is provided. Methods of preparing a variety of packaging is known in the art, including methods of preparing blister packaging.

Another quantitative analysis, one of doctors' quantitative valacyclovir prescribing habits, revealed that the most common prescription of valacyclovir is eight unit doses of 500 mg. Such prescriptions represented more than 28% of all prescriptions written for valacyclovir. The analysis also revealed that the most commonly prescribed quantity of 500 mg of valacyclovir is eight unit doses. Such prescriptions represented more than 30% of all prescriptions written for 500 mg valacyclovir.

The analysis also revealed that the most common prescription written for valacyclovir is a prescription for eight unit doses of 500 mg.

The analysis also revealed that the most common number of unit doses of valacyclovir having a strength of 500 mg prescribed in an individual prescription is eight unit doses.

The analysis also revealed that the most common number of unit doses of valacyclovir having a strength of 1000 mg prescribed in an individual prescription is four unit doses.

The present invention provides a kit comprising precisely eight unit dose compartments, each unit dose compartment comprising precisely one unit dose of 500 mg valacyclovir. Such a kit may combine the advantages of one particular variety of packaging with the advantages of ease of dispensing. For example, eight unit doses of valacyclovir may be provided in a blister sheet thereby providing the benefits of quicker valacyclovir counting, reduced valacyclovir handling, reduced materials required by the pharmacist (e.g. no additional vial is required), increased shelf life, increased patient compliance, reduced spillage and/or other advantages while at the same time reducing the requirement for having to break up the blister sheet. Such a kit will be effective at delivering such advantages in more cases than a kit comprising a number of unit doses of valacyclovir that is not eight.

The analysis of doctors' valacyclovir prescribing habits also revealed that the most commonly prescribed quantity of 1000 mg of valacyclovir is four unit doses. Such prescriptions represented more than 49% of all prescriptions written for 1000 mg valacyclovir.

The present invention also provides a kit comprising precisely four unit dose compartments, each unit dose compartment comprising precisely one unit dose of valacyclovir having a strength selected from the group consisting of 500 mg and 1000 mg wherein each unit dose in each of the four unit dose compartments has the same strength. Such a kit may combine the advantages of one particular variety of packaging with the advantages of ease of dispensing. For example, four unit doses of valacyclovir may be provided in a blister sheet thereby providing the benefits of quicker valacyclovir counting, reduced valacyclovir handling, reduced materials required by the pharmacist (e.g. no additional vial is required), increased shelf life, increased patient compliance, reduced spillage and/or other advantages while at the same time reducing the requirement for having to break up the blister sheet. In the case of a kit having 4 unit doses of 500 mg valacyclovir, such a kit may be combined with another such kit for the purposes of providing two such kits for a total of eight unit doses altogether.

In the event that a prescription is written that comprises a number of unit doses of valacyclovir that is not evenly divisible by four, it may be beneficial to interspace the precisely eight and/or four unit dose compartments by perforations. Such perforations may be provided such that tearing along all of the perforations would result in the physical separation of each of the unit doses from each other. In other words, after tearing the perforations all of the unit doses would be provided in unit dose compartments that are not physically connected. Such perforations may also provide benefits to the consumer of the valacyclovir so that a single dose may be separated from the remaining doses while having all of the doses, separated or not, remain packaged.

In addition to the perforations, it may be beneficial to provided the precisely eight unit dose compartments in four sheets, each one of the four sheets having two unit dose compartments. Furthermore, it may be beneficial to provided unit dose compartments in sheets of two unit dose compartments for the purposes of provided four or eight unit dose kits. The two unit dose compartments may also be interspaced by perforations in the same manner as described above.

The unit dose compartments in a single kit should each contain valacyclovir of the same strength. For example, if one unit dose compartment in the kit contains a unit dose of valacyclovir of 500 mg, then all of the compartments in the kit will contain a unit dose of valacyclovir of 500 mg or be empty. For clarity, according to an illustrative embodiment of the invention, a kit for 500 mg of valacyclovir may comprise eight unit doses of 500 mg valacyclovir and zero unit doses of any other strength. A particular, non-limiting example is a blister sheet having two unit dose cavities each containing a 1000 mg unit dose of valacyclovir and two of such blister sheets (i.e. each sheet having unit doses of 1000 mg in each unit dose cavity) will be in the kit.

The unit dose compartments do not have to be the same size and/or shape. A kit according to the present invention may comprise precisely eight unit dose compartments wherein the size of each of the eight unit dose compartments may be different or the same. Typically, the size and shape of the unit dose compartments are the same and are dependent on the size of the unit dose that is contained within the unit dose compartment. Methods for preparing unit dose compartments having a suitable size and shape for a particular unit dose or unit dosage form are known in the art.

Methods of packaging valacyclovir comprising separating eight unit doses of valacyclovir into eight unit dose compartments, wherein each one of the eight unit dose compartments each holds only one of the eight unit doses of valacyclovir is provided.

Methods of packaging valacyclovir comprising separating four unit doses of valacyclovir into four unit dose compartments, wherein each one of the four unit dose compartments each holds only one of the four unit doses of valacyclovir is provided. Methods of preparing a variety of packaging is known in the art, including methods of preparing blister packaging.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A method of packaging one or more pharmaceuticals comprising separating a plurality of unit doses into a plurality of unit dose compartments, wherein each unit dose compartment of the plurality of unit dose compartments holds only one unit dose of the plurality of unit doses, each unit dose having the same strength, wherein the plurality of unit doses is precisely a quantity equal to a most commonly prescribed number of unit doses in individual prescriptions for the unit dose, or a multiple thereof.

2. The method of claim 1 wherein the plurality of unit doses is precisely the quantity equal to the most commonly prescribed number of unit doses in individual prescriptions for the pharmaceutical.

3. The method of claim 1 further comprising labeling each unit dose compartment.

4. The method of claim 1 wherein each unit dose compartment is a blister cavity.

5. The method of claim 1 further comprising obtaining quantitative prescribing information for the one or more pharmaceuticals prior to separating the plurality of doses.

6. A method of designing packaging for one or more pharmaceuticals comprising obtaining quantitative prescribing information for the one or more pharmaceuticals; and preparing a plurality of unit dose compartments operable to hold only one unit dose and the plurality of unit dose compartments is precisely a quantity equal to a most commonly prescribed number of unit doses in individual prescriptions for the unit dose, or a multiple thereof.

7. The method of claim 6 wherein the quantitative prescribing information is based on the same one or more pharmaceuticals that comprise each unit dose.

8. The method of claim 5 wherein the quantitative prescribing information is based on one or more pharmaceuticals that are comparable to each unit dose.

9. A kit comprising a plurality of unit doses in a plurality of unit dose compartments, wherein each unit dose compartment of the plurality of unit dose compartments holds only one unit dose of the plurality of unit doses, each unit dose having the same strength, wherein the plurality of unit doses is precisely a quantity equal to a most commonly prescribed number of unit doses in individual prescriptions for the pharmaceutical, or a multiple thereof.

10. The kit of claim 9 wherein the plurality of unit doses is precisely the quantity equal to the most commonly prescribed number of unit doses in individual prescriptions for the pharmaceutical.

11. The kit, of claim 9 wherein each unit dose compartment further comprises a label.

12. The kit of claim 9 wherein each unit dose compartment is a blister cavity.

13. A kit comprising precisely 30 unit dose compartments, each unit dose compartment comprising precisely one unit dose of atorvastatin having a strength selected from the group consisting of 1.0 mg, 20 mg and 40 mg wherein each unit dose in each of the 30 unit dose compartments has the same strength.

14. The kit of claim 13 wherein the precisely 30 unit dose compartments are interspaced by perforations.

15. The kit of claim 13 wherein the precisely 30 unit dose compartments are blister cavities.

16. The kit of claim 13 wherein the precisely 30 unit dose compartments comprise three sheets, each sheet having 10 unit dose compartments.

17. The kit of claim 16 wherein the 10 unit dose compartments on each sheet are interspaced by perforations.

18. The kit of claim 16 wherein the 10 unit dose compartments on each sheet are blister cavities.

19. The kit of claim 13 wherein the strength of each unit dose of atorvastatin is 10 mg.

20. The kit of claim 13 to wherein the strength of each unit dose of atorvastatin is 20 mg.

21. The kit of claim 13 wherein the strength of each unit dose of atorvastatin is 40 mg.

22. A method of packaging atorvastatin comprising separating 30 unit doses of atorvastatin into 30 unit dose compartments, wherein each one of the 30 unit dose compartments each holds only one of the 30 unit doses of atorvastatin, each unit dose of atorvastatin having a strength selected from the group consisting of 10 mg, 20 mg and 40 mg and each unit dose of atorvastatin dose having the same strength.

23. The method of claim 22 wherein the 30 unit dose compartments are 30 blister cavities.

24. The method of claim 23 wherein the 30 blister cavities are further packaged inside a box.

25. The method of claim 22 wherein the strength of each unit dose of atorvastatin is 10 mg.

26. The method of claim 22 wherein the strength of each unit dose of atorvastatin is 20 mg.

27. The method of claim 22 wherein the strength of each unit dose of atorvastatin is 40 mg.

28. A kit comprising precisely eight unit dose compartments, each unit dose compartment comprising precisely one unit dose of 500 mg valacyclovir.

29. The kit of claim 28 wherein the precisely eight unit dose compartments are interspaced by perforations.

30. The kit of claim 28 wherein the precisely eight unit dose compartments comprise four sheets, each sheet having two unit dose compartments.

31. The kit of claim 30 wherein the two unit dose compartments on each sheet are interspaced by perforations.

32. The kit of claim 28 wherein the unit dose compartments are blister cavities.

33. A kit comprising precisely four unit dose compartments, each unit dose compartment comprising precisely one unit dose of valacyclovir having a strength selected from the group consisting of 500 mg and 1000 mg wherein each unit dose in each of the four unit dose compartments has the same strength.

34. The kit of claim 33 wherein the precisely four unit dose compartments are interspaced by perforations.

35. The kit of claim 33 wherein the precisely four unit dose compartments comprise two sheets, each sheet having two unit dose compartments.

36. The kit of claim 35 wherein the two unit dose compartments on each sheet are interspaced by perforations.

37. The kit of claim 33 wherein the unit dose compartments are blister cavities.

38. The kit of claim 33 wherein the strength of each unit dose of valacyclovir is 500 mg.

39. The kit of claim 33 wherein the strength of each unit dose of valacyclovir is 1000 mg.

40. A method of packaging valacyclovir comprising separating eight unit doses of 500 mg valacyclovir into eight unit dose compartments wherein each one of the eight unit dose compartments each holds only one of the eight unit doses of 500 mg valacyclovir.

41. The method of claim 40 wherein the eight unit dose compartments are eight blister cavities.

42. The method of claim 41 wherein the eight blister cavities are further packaged inside a box.

43. A method of packaging valacyclovir comprising separating four unit doses of valacyclovir into four unit dose compartments wherein each one of the four unit dose compartments each holds only one of the four unit doses of valacyclovir, each unit dose of valacyclovir having a strength selected from the group consisting of 500 mg and 1000 mg and each unit of valacyclovir dose having the same strength.

44. The method of claim 43 wherein the four unit dose compartments are four blister cavities.

45. The method of claim 44 wherein the four blister cavities are further packaged inside a box.

46. The method of claim 43 wherein the strength of each unit dose of valacyclovir is 500 mg.

47. The method of claim 43 wherein the strength of each unit dose of valacyclovir is 1000 mg.
